(19) United States
(12) Patent Application Publication
(10) Pub. No.: US 2023/0190821 A1
(43) Pub. Date: Jun. 22, 2023

(54) REGENERATIVE NONSTEROIDAL ANTI-INFLAMMATORY COMPOSITIONS, METHODS OF PRODUCTION, AND METHODS OF USE THEREOF

(71) Applicant: CROWN SCIENTIFIC, L.L.C., San Antonion, TX (US)

(72) Inventor: Ramon Coronado, San Antonion, TX (US)

(21) Appl. No.: 17/919,453

(22) PCT Filed: Apr. 16, 2021
(86) PCT No.: PCT/US2021/027674

§ 371 Date: Oct. 17, 2022

Related U.S. Application Data
(60) Provisional application No. 63/011,373, filed on Apr. 17, 2020.

(51) Int. Cl. A61K 35/50; A61K 35/28; A61K 31/557; A61P 37/06; A61P 9/00; A61P 17/00; A61P 19/04; A61P 31/14; A61P 11/00; A61K 45/06; C12N 5/0735

(52) U.S. Cl. CPC A61K 35/50; A61K 35/28; A61K 31/557; A61P 37/06; A61P 9/00; A61P 17/00; A61P 19/04; A61P 31/14; A61P 11/00; A61K 45/06; C12N 5/0606; C12N 2500/02

(57) ABSTRACT

The disclosure provides nonsteroidal anti-inflammatory compositions and methods of use thereof. Specifically, the disclosure provides cell-free or substantially cell-free regenerative nonsteroidal anti-inflammatory compositions derived from placenta and/or from MSC cells isolated therefrom, methods for producing said compositions, and uses thereof to treat chronic and acute inflammatory conditions and diseases.

### RELATED APPLICATIONS

This application relates to and claims priority to U.S. Provisional Application No. 63/011,373, filed on Apr. 17, 2020, the contents of which are incorporated by reference in their entirety.

### FIELD OF THE ART

The present disclosure generally relates to nonsteroidal anti-inflammatory compositions, and more particularly, to cell-free or substantially cell-free regenerative nonsteroidal anti-inflammatory compositions derived from placenta and/or from MSC cells isolated therefrom, methods for producing said compositions, and uses thereof to treat chronic and acute inflammatory conditions and diseases.

### BACKGROUND

The World Health Organization ranks chronic inflammatory diseases as the greatest threat to human health. Worldwide, three out of five people die due to chronic inflammatory diseases. In the United States, 60% of people have one inflammatory disease or condition, 42% of people have more than one inflammatory disease or condition, and 12% of people have more than five inflammatory diseases or conditions.

Currently, inflammation is often treated with steroids as well as nonsteroidal anti-inflammatory drugs (NSAIDs). But both steroids and NSAIDs are cytotoxic and therefore inhibit healing and regeneration.

Stem cell therapy is an emerging therapeutic approach for treating inflammation. Although stem cells reduce inflammation and also promote healing, stem cell therapy has numerous hurdles. For example, protecting stem cell intellectual property and regulating stem cells for therapeutic commercial use remains ambiguous and highly complex. Further, living stem cells must remain frozen, which increases costs and complicates logistics for storage and distribution. Additionally, there are issues related to determining the appropriate dosage of live cells, especially considering that a portion of the cells may have died prior to administration to a patient. This problem is exacerbated by the fact that there is little to no validation of claims to having the best stem cell technology or the most living cells in a given product in today's saturated stem cell market.

### BRIEF SUMMARY

The present disclosure generally encompasses a cell-free or substantially cell-free regenerative nonsteroidal anti-inflammatory (RNSA) composition suitable for therapeutic or prophylactic use comprising a therapeutically or prophylactically effective amount of an isolated cell-free or substantially cell-free placenta-derived extract obtained from placental tissue from one or more mammalian donors wherein such tissue has naturally or been induced to undergo apoptosis or controlled cell death, wherein said extract may comprise one or more eicosanoids optionally selected from 6kPGF1α, TXB2, PGF2α, PGE2, PGA2, LTB4, 5oxoETE, 5HETE, 11HETE, 12HETE, 15HETE, 20HETE, 5,6DHET, 8,9DHET, 11,12DHET, 14,15DHET, 9HODE, 13HODE, and AA, wherein said composition optionally is capable of inhibiting proliferation of activated T cells and/or is non-cytotoxic for one or more cells selected from stromal cells, mesenchymal stromal cells (MSCs), parenchymal cells, and tenocytes in a subject in need thereof, in vivo, or in vitro.

In some embodiments, the placenta may be selected from human, non-human primate, pig, sheep, horse, cow, dog, cat, rat, and mouse placenta. In some embodiments, the placenta may preferably be human placenta.

In some embodiments, the placental tissue may be obtained from a single donor.

In some embodiments, the placental tissue may be obtained from more than one donor (pooled donor placental tissue sample).

In some aspects, the placenta may comprise at least one placental tissue selected from amniotic membrane, chorion membrane, chorionic villus, umbilical cord, and Wharton's Jelly. The placenta may preferably be selected from at least one of amniotic membrane and/or chorion membrane.

In some embodiments, the at least one placental tissue may comprise perinatal stromal cells (PSCs) and/or mesenchymal stromal cells (MSCs).

In certain embodiments, the RNSA composition may be stable in solution at room temperature for at least eight weeks.

In certain embodiments, the RNSA composition may be stable to lyophilization.

In some embodiments, the RNSA composition may be capable of inhibiting proliferation of activated T cells, wherein the T cells are CD4+, CD8+, CD4+/CD8+, CD11c+, CD11b+, and/or CD56+ T cells.

In some embodiments, the composition may be further capable of promoting proliferation of one or more cells selected from stromal cells, mesenchymal stromal cells (MSCs), parenchymal cells, and tenocytes in a subject, in vivo, or in vitro.

In some embodiments, the RNSA composition may be capable of reducing expression of one or more pro-inflammatory cytokines from activated peripheral blood mononucleated cells (PBMCs) and/or activated T cells in a subject, in vivo, or in vitro. The one or more pro-inflammatory cytokines may be selected from TNFα, NFκB, IL-17A, IL-6, and IFNγ.

In some embodiments, the RNSA composition may be capable of increasing cAMP production from activated T cells in a subject, in vivo, or in vitro.

Moreover, the present disclosure also generally encompasses a method for producing a cell-free or substantially cell-free regenerative nonsteroidal anti-inflammatory (RNSA) composition. The method may comprise (i) obtaining at least one placental tissue from at least one mammal selected from human, non-human primate, pig, sheep, horse, cow, dog, cat, rat, and mouse, wherein the at least one placental tissue is selected from amniotic membrane, chorion membrane, chorionic villus, umbilical cord, and Wharton's Jelly, and wherein the at least one placental tissue comprises perinatal stromal cells (PSCs); (ii) optionally isolating the PSCs from said placental tissue and culturing the PSCs in at least one cell culture medium; (iii) permitting apoptosis of said placental tissue and PSCs comprised therein and/or permitting apoptosis of PSCs isolated therefrom to naturally occur and/or inducing apoptosis of said placental tissue and PSCs comprised therein and/or inducing apoptosis of PSCs isolated therefrom to produce an apoptotic extract; and (iv) separating the apoptotic extract or a portion thereof from the cells and tissue, for example, by decantation, centrifugation, and/or filtration; thereby producing the cell-free or substantially cell-free regenerative nonsteroidal anti-inflammatory composition.

In some embodiments, the PSCs may comprise mesenchymal stromal cells (MSCs).

In some embodiments, the mammal may be a human.

In some embodiments, the method may further comprise conducting one or more screening assays to assess the effects of the isolated apoptotic extract or one or more portions thereof on the proliferation of activated T cells and/or the proliferation of one or more cells selected from stromal cells, mesenchymal stromal cells (MSCs), parenchymal cells, and tenocytes and/or on the expression of pro-inflammatory cytokines and/or the expression of anti-inflammatory cytokines in a mammalian subject or in vitro.

In some embodiments, different portions of the isolated apoptotic extract may be screened in order to assess potency.

In some embodiments, inducing apoptosis may comprise serum deprivation, nutrient deprivation, and/or hypoxia.

In some embodiments, inducing apoptosis may comprise (i) contacting the placental tissue with a non-cell culture medium in a ratio ranging from about 1 mL non-cell culture medium per 1 gram of placental tissue to about 100 mL non-cell culture medium per 1 gram of placental tissue, preferably in a ratio of about 10 mL non-cell culture medium per 1 g of placental tissue; and (ii) incubating the placental tissue in the non-cell culture medium in an air-tight environment at a temperature ranging from about 4° C. to about 42° C., preferably at about 37° C., for about 2 days to about 12 days, preferably for about 10 days, wherein the incubating optionally comprises agitation, for example, at about 90 rpm.

In some aspects, the method may further comprise isolating the placental tissue PSCs and culturing the PSCs in at least one cell culture medium prior to inducing apoptosis, optionally by nutrient deprivation and/or hypoxic conditions.

In some embodiments, inducing apoptosis may comprise (i) replacing the at least one cell culture medium with a non-cell culture medium; and (ii) incubating the cultured MSCs in the non-cell culture medium in an air-tight environment at a temperature ranging from about 4° C. to about 42° C., preferably at about 37° C., for about 3 days to about 5 days, preferably for about 4 days, wherein the incubating optionally comprises agitation.

In some embodiments, the cultured PSCs may be cultured to at least 80% confluence.

In some embodiments, the non-cell culture medium may comprise saline solution.

In some embodiments, the saline solution may comprise 0.9% NaCl.

In some embodiments, the saline solution may comprise phosphate-buffered saline. (PBS).

In some embodiments, the air-tight environment may prevent gas exchange, thereby inducing a hypoxic environment.

In some embodiments, the method may further comprise washing the placental tissue with phosphate-buffered saline (PBS) prior to inducing apoptosis.

In some embodiments, the method may further comprise mincing the placental tissue prior to inducing apoptosis.

In some embodiments, the method may further comprise washing the cultured MSCs with phosphate-buffered saline (PBS) prior to inducing apoptosis.

In some embodiments, the method may further comprise contacting the placental tissue with one or more antimicrobial agents.

In some embodiments, the method may further comprise centrifugation at about 10,000×g for about 30 minutes.

In some embodiments, the method may further comprise filtration through a 0.45 μm membrane.

In some embodiments, the method may further comprise filtration through a 0.2 μm membrane, i.e. sterile filtration.

In some embodiments, the method may further comprise filtration through a 30 KDa MWCO membrane, a 10 KDa MWCO membrane, a 5 KDa MWCO membrane, a 3 KDa MWCO membrane, and/or a 2 KDa MWCO membrane.

Moreover, the present disclosure also generally relates to a cell-free or substantially cell-free regenerative nonsteroidal anti-inflammatory (RNSA) composition produced by the methods described herein.

In some embodiments, the composition may comprise one or more eicosanoids optionally selected from 6kPGF1α, TXB2, PGF2α, PGE2, PGA2, LTB4, 5oxoETE, 5HETE, 11HETE, 12HETE, 15HETE, 20HETE, 5,6DHET, 8,9DHET, 11,12DHET, 14,15DHET, 9HODE, 13HODE, and AA.

In some embodiments, the composition may be capable of inhibiting proliferation of activated T cells, wherein the T cells are CD4+, CD8+, CD4+/CD8+, CD11c+, CD11b+, and/or CD56+ T cells in a subject, in vivo, or in vitro.

In some embodiments, the composition may be non-cytotoxic for one or more cell types selected from stromal cells, mesenchymal stromal cells (MSCs), parenchymal cells, and tenocytes in a subject, in vivo, or in vitro.

In some embodiments, the composition may be capable of promoting proliferation of one or more cell types selected from stromal cells, mesenchymal stromal cells (MSCs), parenchymal cells, and tenocytes in a subject, in vivo, or in vitro.

In some embodiments, the composition may be capable of reducing expression of one or more pro-inflammatory cytokines from activated peripheral blood mononucleated cells (PBMCs) and/or activated T cells in a subject, in vivo, or in vitro.

In some embodiments, the one or more pro-inflammatory cytokines may be selected from TNFα, NFκB, IL17A, IL-6, and IFNγ.

In some embodiments, the composition may be capable of increasing cAMP production from activated T cells in a subject, in vivo, or in vitro.

In some embodiments, the composition may be stable in solution at room temperature for at least eight weeks.

In some embodiments, the composition may be stable to lyophilization.

The present disclosure also encompasses a method of treatment or prevention of at least one inflammatory condition or disease or at least one symptom associated therewith, comprising administering a therapeutically or prophylactically effective amount of the cell-free or substantially cell-free regenerative nonsteroidal anti-inflammatory (RNSA) composition of any one of the foregoing claims to a subject in need thereof.

In some embodiments, the at least one inflammatory condition or disease may be an acute or chronic condition associated with inflammation, e.g., an acute or chronic autoimmune disease associated with acute or chronic inflammation, optionally a viral or bacterial or fungal infection associated with acute or chronic inflammation, further optionally a hepatitis virus, ZIKA virus, herpes, papillomavirus, influenza virus, or coronavirus, further optionally COVID-19 or SARS.

In some embodiments, the at least one inflammatory condition or disease may be an acute inflammatory condition or disease optionally a viral infection associated with acute inflammation, further optionally a coronavirus infection, e.g., COVID-19 or SARS.

In some embodiments, the at least one inflammatory condition or disease may be selected from pneumonia, single or multiple organ failure or dysfunction, sepsis, cytokine storm, fever, neurological dysfunction or impairment, loss of taste or smell, cardiac dysfunction, pulmonary dysfunction, liver dysfunction, acute or chronic respiratory dysfunction, graft versus host disease (GVHD), cardiomyopathy, vasculitis, fibrosis, ophthalmic inflammation, dermatologic inflammation, gastrointestinal inflammation, tendinopathies, allergy, asthma, glomerulonephritis, pancreatitis, hepatitis, inflammatory arthritis, gout, multiple sclerosis, psoriasis, Acute Respiratory Distress Syndrome (ARDS), wound healing, diabetic ulcers, non-healing wounds, lupus, and other autoimmune diseases associated with acute or chronic inflammation.

In some embodiments, the symptoms associated with the inflammatory condition may include one or more of pneumonia, cytokine storm, single or multiple organ failure, fibrosis, impaired respiratory function such as acute or chronic respiratory distress syndrome, fever, impaired cardiac function, impaired lung function, impaired liver function, impaired taste or smell, and impaired neurological function.

In exemplary embodiments, the subject may have pneumonia, optionally Covid-19-associated pneumonia and/or a pneumonia associated with another virus, e.g., influenza or another coronavirus, and/or a pneumonia associated with a fungus or bacterium.

In some embodiments, the ophthalmic inflammation may comprise one or more of corneal regeneration, corneal wound healing, corneal melting, dry eye, ocular infection, eyelid sty, and autoimmune-associated peripheral ulcerative keratitis.

In some embodiments, the fibrosis may comprise one or more of pulmonary fibrosis, cystic fibrosis, idiopathic pulmonary fibrosis, interstitial pulmonary fibrosis, radiation-induced lung injury, liver fibrosis, bridging fibrosis of the liver, cirrhosis, glial scar, arterial stiffness, arthrofibrosis, Crohn's disease, Dupuytren's contracture, keloid fibrosis, Mediastinal fibrosis, Myelofibrosis, Myocardial fibrosis, Peyronie's disease, Nephrogenic systemic fibrosis, Progressive massive fibrosis, pneumoconiosis, Retroperitoneal fibrosis, stromal fibrosis, Scleroderma, systemic sclerosis, Chronic obstructive pulmonary disease (COPD), asthma, and adhesive capsulitis.

In some embodiments, the gastrointestinal inflammation may comprise one or more of inflammatory bowel disease (IBD), Crohn's disease, ulcerative colitis, irritable bowel syndrome (IBS), and Celiac disease.

In some embodiments, the ophthalmic inflammation may be associated with keratoconjunctivitis sicca.

In some embodiments, the dermatologic inflammation may comprise eczema and psoriasis.

In some embodiments, the at least one autoimmune disease may be selected from the group consisting of Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome, (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndrome type I, Polyglandular syndrome type II, Polyglandular syndrome type III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, and Vogt-Koyanagi-Harada Disease.

In exemplary embodiments, the effective amount may comprise one or more doses of the composition. Each dose may range from 0.1 mL/10 kg body weight to 10 mL/10 kg body weight, preferably 1 mL/10 kg body weight.

In some embodiments, the composition may be administered by one or more of injection, optionally intravenous (IV), subcutaneous (SC) administration, nebulization, and eye drops.

In some embodiments, the subject may be selected from a human, non-human primate, pig, sheep, horse, cow, dog, cat, rat, and mouse. In preferred embodiments, the subject may be human.

In some embodiments, the method of treatment or prevention may further comprise the administration of at least one other active, e.g., an anti-inflammatory agent such as an anti-inflammatory antibody or anti-inflammatory fusion protein, an antiviral agent, an antibacterial agent, an antifungal agent, an analgesic, an anti-congestive agent, an anti-fever agent, or a combination of any of the foregoing.

In exemplary embodiments, the subject may have been diagnosed with or is suspected of having a coronavirus infection, optionally COVID-19.

In some embodiments, the subject may have been diagnosed with a coronavirus infection, optionally COVID-19, and is on a respirator, has Acute Respiratory Distress Syndrome, and/or is experiencing respiratory difficulties.

In some embodiments, the subject may have been diagnosed with or suspected of having a coronavirus infection, optionally COVID-19, and optionally the subject comprises one or more risk factors that place the subject at higher risk for morbidity or a poor treatment outcome, e.g., age over 55 years, obesity, diabetes, cardiac problem or condition, respiratory condition, optionally asthma, COPD, cystic fibrosis, is a smoker, is a heavy drinker, has lupus, has elevated blood pressure, has cancer, receives chemotherapy, has (chronic) kidney disease and/or is on dialysis, or any combination of the foregoing.

### DESCRIPTION OF THE DRAWINGS

FIG. 1A illustrates an exemplary method for preparing human placenta for isolation of amniotic membrane.

FIG. 1B illustrates an exemplary method for peeling or removing amniotic membrane from chorion.

FIG. 1C illustrates an exemplary method for washing amniotic membrane with PBS.

FIG. 2 presents data showing normalized activated T cell proliferation as a model for inflammation and inhibition of the T cell proliferation of greater than 85% by MSCs (positive control). Results for cell-free extracts prepared from nineteen conditions (C1-C19, various tissues and conditions for treating those tissues) are also shown. C14 represents the cell-free regenerative nonsteroidal anti-inflammatory composition (RNSA) described in Example 1.

FIG. 3 presents data showing normalized activated T cell proliferation as a model for inflammation. It can be seen therefrom that the cell-free regenerative nonsteroidal anti-inflammatory composition described in Example 1 (RNSA), inhibited T cell proliferation by greater than 85%, comparable to the T cell proliferation inhibition achieved by MSCs, whereas the control, (commercial cell-free reagent) did not inhibit activated T cell proliferation.

FIG. 4 presents data showing activated T cell proliferation as a model for inflammation and results from testing different methods of cell death on the potency of the cell-free RNSA composition.

FIG. 5 presents data showing activated T cell proliferation as a model for inflammation and results from testing various filtration membranes on the potency of the cell-free RNSA composition.

FIG. 6 presents data showing activated T cell proliferation as a model for inflammation and results from testing the effect of lyophilization on the potency of the RNSA compositions.

FIG. 7 presents data showing activated T cell proliferation as a model for inflammation and results from testing the effect of DNase, RNase, and Proteinase K on the potency of the RNSA composition produced from amniotic membrane tissue extraction described in Example 1.

FIG. 8 presents data showing activated T cell proliferation as a model for inflammation and results from testing the effect of DNase, RNase, and Proteinase K on the potency of the RNSA composition produced from cultured hMSCs described in Example 2.

FIG. 9 presents data showing activated T cell viability as a model for inflammation and results from testing the effect of DNase, RNase, and Proteinase 1<on the potency of the RNSA composition produced from amniotic membrane tissue extraction described in Example 1.

FIG. 10 presents data showing activated T cell proliferation as a model for inflammation and results from testing the effect of time during extraction and shaking vs. non-shaking conditions on the potency of the RNSA composition produced from amniotic membrane tissue extraction described in Example 1.

FIG. 11 presents data showing activated T cell proliferation as a model for inflammation and results from testing the effect of tissue type and culture media on the potency of the RNSA composition produced from tissue extractions described in Example 1.

FIG. 12 presents data showing activated T cell proliferation as a model for inflammation and results from testing the stability of the RNSA compositions stored at room temperature (RT) and at 4° C. for two months.

FIG. 13 presents data showing activated T cell proliferation as a model for inflammation and results from testing the effect of EP Receptor blockers on the potency of the RNSA compositions.

FIG. 14 presents data showing activated CD4+ T cell proliferation within a PBMC sample as a model for inflammation and results from testing the effect of the tissue extraction process described in Example 1 (“AM”) compared to the cultured MSCs extraction process described in Example 2 (“BR”) on the potency of the RNSA compositions.

FIG. 15 presents data showing activated CD8+ T cell proliferation within a PBMC sample as a model for inflammation and results from testing the effect of the tissue extraction process described in Example 1 (“AM”) compared to the cultured MSCs extraction process described in Example 2 (“BR”) on the potency of the RNSA compositions.

FIG. 16 presents data showing activated CD4+/CD8+ T cell proliferation within a PBMC sample as a model for inflammation and results from testing the effect of the tissue extraction process described in Example 1 (“AM”) compared to the cultured MSCs extraction process described in Example 2 (“BR”) on the potency of the RNSA compositions.

FIG. 17 presents data showing activated CD11c+ T cell proliferation within a PBMC sample as a model for inflammation and results from testing the effect of the tissue extraction process described in Example 1 (“AM”) compared to the cultured MSCs extraction process described in Example 2 (“BR”) on the potency of the RNSA compositions.

FIG. 18 presents data showing activated CD11b+ T cell proliferation within a PBMC sample as a model for inflammation and results from testing the effect of the tissue extraction process described in Example 1 (“AM”) compared to the cultured MSCs extraction process described in Example 2 (“BR”) on the potency of the RNSA compositions.

FIG. 19 presents data showing activated CD56+ T cell proliferation within a PBMC sample as a model for inflammation and results from testing the effect of the tissue extraction process described in Example 1 (“AM”) compared to the cultured MSCs extraction process described in Example 2 (“BR”) on the potency of the RNSA compositions.

FIG. 20 shows that the RNSA composition reduces the expression level of TNFα from activated PBMCs.

FIG. 21 shows that the RNSA composition reduces the expression level of NFκB from activated PBMCs.

FIG. 22 shows that the RNSA composition reduces the expression level of IL-17A from activated PBMCs.

FIG. 23 shows that the RNSA composition reduces the expression level of IFNγ from activated PBMCs.

FIG. 24 shows that the RNSA composition promotes/induces cAMP production by activated T cells.

FIG. 25 shows results of the eicosanoid analysis of RNSA compositions.

FIG. 26 shows a list of eicosanoids which were not detected in the eicosanoid analysis of RNSA compositions.

FIG. 27 shows proliferation of human stromal cells as a model for regeneration and promotion of stromal cell proliferation by the RNSA composition. In contrast, the steroid is completely cytotoxic.

FIG. 28 shows proliferation of hMSCs as a model for regeneration and promotion of hMSC proliferation by the RNSA composition. In contrast, the steroid is completely cytotoxic.

FIG. 29 shows proliferation of human parenchymal cells as a model for regeneration and promotion of human parenchymal cells proliferation by the RNSA composition. In contrast, the tested commercial compounds were each cytotoxic to varying degrees.

FIG. 30 shows proliferation of human tenocytes as a model for regeneration and promotion of human tenocytes proliferation by the RNSA composition. In contrast, the tested commercial compounds were each cytotoxic to varying degrees.

FIG. 31 shows data relating to survival proportions for a mouse model of Graft versus Host Disease.

FIG. 32 is a plot of the GvHD score versus normalized days for the GvHD mice injected with media (PBMC) or with an exemplary cell-free regenerative nonsteroidal anti-inflammatory composition (Cell-Free) according to the invention.

FIG. 33 is a plot of the body weight GvHD mice injected with media (PBMC) or with an exemplary cell-free regenerative nonsteroidal anti-inflammatory composition (CM) according to the invention.

FIG. 34A is a photo of GvHD mouse model kidneys.

FIG. 34B is a plot of the GvHD mouse kidney areas in mm².

FIG. 35 is a photo of the GvHD mice. A GvHD mouse treated with an exemplary RNSA composition according to the invention is shown on the left whereas an untreated GvHD mouse is shown on the right.

FIG. 36 shows plots quantifying the levels of inflammatory markers IL-17 and IFNγ at Day 21 and Day 42 in a mouse model of cardiomyopathy.

FIG. 37 shows plots quantifying the inflammatory markers histopathology disease scores (H&E) and the Trichrome fibrosis scores at Day 21 in a mouse model of cardiomyopathy.

FIG. 38 shows a photo of a human patient having eczema on the left hand on Day 1 and a photo of the same on Day 60 after a first subcutaneous injection of an exemplary RNSA composition according to the invention on Day 1 and a second subcutaneous injection of the same RNSA composition on Day 30.

FIG. 39 shows an ultrasound image of a human ankle tendinosis prior to treatment (subcutaneous injection of an exemplary RNSA composition according to the invention at the site of tendinosis) and an ultrasound image of the healed tendon 30 days after treatment.

FIG. 40 shows two photos of a human patient having an eyelid sty prior to treatment with eye drops comprising an exemplary RNSA composition according to the invention (top) and two photos of the same eye six weeks after treatment showing that the sty had completely healed (bottom).

### DETAILED DESCRIPTION

### I. Overview

Provided herein are cell-free (or substantially cell-free) regenerative nonsteroidal anti-inflammatory compositions derived from placenta, methods for producing said compositions, and uses thereof to treat chronic and acute inflammatory conditions and diseases.

It is contemplated that any embodiment discussed in this specification can be implemented with respect to any method, kit, reagent, or composition of the disclosure, and vice versa. Furthermore, compositions of this disclosure can be used to achieve methods of the disclosure.

It will be understood that particular embodiments described herein are shown by way of illustration and not as limitations. The principal features of this disclosure can be employed in various embodiments without departing from the scope of the disclosure. Those skilled in the art will recognize, or be able to ascertain, using no more than routine experimentation, numerous equivalents to the specific procedures described herein. Such equivalents are considered to be within the scope of this disclosure and are covered by the appended claims.

All publications and patent applications mentioned in the instant specification are indicative of the level of skill of one skilled in the art to which this disclosure pertains. All publications and patent applications are herein incorporated by reference to the same extent as if each individual publication or patent application was specifically and individually indicated to be incorporated by reference.

Unless defined otherwise, all technical and scientific terms used herein have the same meaning as is commonly understood by one of skill in the art to which this disclosure belongs. In the event that there are a plurality of definitions for terms herein, those in this section prevail. Where reference is made to a URL or other such identifier or address, it is to be understood that such identifiers can change and particular information on the internet can come and go, but equivalent information can be found by searching the internet. Reference thereto evidences the availability and public dissemination of such information.

As used herein, the singular forms “a,” “an,” and “the” may mean “one” but also include plural referents such as “one or more” and “at least one” unless the context clearly dictates otherwise. All technical and scientific terms used herein have the same meaning as commonly understood to one of ordinary skill in the art to which this invention belongs unless clearly indicated otherwise.

As used herein, the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the inherent variation of error for the device, the method being employed to determine the value, or the variation that exists among the study subjects.

As used herein, words of approximation such as, without limitation, “about,” “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 8, 9, 10, 11, 12, 13, 14, or 15%.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, “apoptosis” refers to programmed cell death via a highly-regulated, genetically-directed process of cell self-destruction that is marked by the fragmentation of nuclear DNA, is activated either by the presence of a stimulus or removal of a suppressing agent or stimulus and is a normal physiological process.

As used herein “cell-free” generally refers to a composition or extract, e.g., a placental derived extract, wherein all cells originally contained in the composition or extract have been removed or rendered non-viable. In the present invention this is generally achieved by inducing apoptosis such as by use of nutrient deprivation and removal of live and/or apoptosed cells such as by the use of decantation, centrifugation, and/or filtration.

As used herein “substantially cell-free” generally refers to a composition or extract, e.g., a placental derived extract, wherein the majority of the cells originally contained in the composition or extract have been removed or rendered non-viable, e.g., wherein at least 70, 80, 90, 95, 99, 99.5, or 99.9% of the cells have been removed or rendered non-viable. In the present invention this is generally achieved by inducing apoptosis such as by the use of nutrient deprivation and removal of live and/or apoptosed cells such as by the use of decantation, centrifugation, and/or filtration.

As used herein “non-cell culture medium” generally refers to a medium wherein cells are cultured that lacks cells and which moreover may lack nutrients which may induce nutrient deprivation, apoptosis, and/or hypoxia of cells contained therein, e.g., a saline medium or composition.

As used herein, “treatment” (and grammatical variations thereof such as “treat” or “treating”) or “prevention” (and grammatical variations thereof such as “prevent” or “preventing”) refers to complete or partial amelioration or reduction of a disease or condition or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply necessarily complete curing of a disease or complete elimination of any symptom or effect(s) on all symptoms or outcomes.

An “effective amount” of an agent, e.g., a pharmaceutical formulation, cells, or composition, in the context of administration, refers to an amount effective, at dosages/amounts and for periods of time necessary, to achieve a desired result, such as a therapeutic or prophylactic result alone or in combination with other active agents.

A “therapeutically effective amount” of an agent, e.g., a pharmaceutical formulation or cells, refers to an amount effective, at dosages and for periods of time necessary, to achieve a desired therapeutic result, such as for treatment of a disease, condition, or disorder, and/or pharmacokinetic or pharmacodynamic effect of the treatment. Generally, the response is either amelioration of symptoms in a patient or a desired biological outcome (e.g., reduction of tissue fibrosis, reduction of tissue inflammation, increase of immune modulation). The therapeutically effective amount may vary according to factors such as the disease state, age, sex, and weight of the subject. In some embodiments, the provided methods involve administering the compositions at effective amounts, e.g., therapeutically effective amounts alone or in combination with other active agents or therapies.

A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount will be less than the therapeutically effective amount. In the context of lower disease burden, the prophylactically effective amount in some aspects will be higher than the therapeutically effective amount.

By “pharmaceutically acceptable” it is meant that the carrier, diluent or excipient must be compatible with the other ingredients of the formulation and not deleterious to the recipient thereof. Pharmaceutically acceptable carriers, excipients or stabilizers are well known in the art, for example Remington's Pharmaceutical Sciences, 16th edition, Osol, A. Ed. (1980). Pharmaceutically acceptable carriers, excipients, or stabilizers are nontoxic to recipients at the dosages and concentrations employed, and may include buffers such as phosphate, citrate, and other organic acids; antioxidants including ascorbic acid, vitamin A, vitamin E, and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, cysteine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (for example, Zn-protein complexes); and/or non-ionic surfactants such as TWEEN™, PLURONICS™ or polyethylene glycol (PEG); retinyl palmitate, selenium, methionine, citric acid, sodium sulfate and parabens Examples of diluent include, but are not limited to, water, alcohol, saline solution, glycol, mineral oil and dimethyl sulfoxide (DMSO).

The pharmaceutical composition may also contain other therapeutic agents, and may be formulated, for example, by employing conventional vehicles or diluents, as well as pharmaceutical additives of a type appropriate to the mode of desired administration (for example, excipients, preservatives, etc.) according to techniques known in the art of pharmaceutical formulation. The pharmaceutical composition may further contain additional pharmaceutical or therapeutic agent, as evaluated beneficial by the physician administering said pharmaceutical composition.

The term “subject” as used herein refers to any individual or patient to which the subject methods are performed. Generally, the subject is human, although as will be appreciated by those in the art, the subject may be an animal. Thus, other animals, including vertebrate such as rodents (including mice, rats, hamsters and guinea pigs), cats, dogs, rabbits, farm animals including cows, horses, goats, sheep, pigs, chickens, etc., and non-human primates (including monkeys, chimpanzees, orangutans and gorillas) are included within the definition of subject.

The terms “administration of” and or “administering” should be understood to mean providing a pharmaceutical composition in a therapeutically effective amount to the subject in need of treatment. Administration routes can be enteral, topical or parenteral. As such, administration routes include but are not limited to intracutaneous, subcutaneous, intravenous, intraperitoneal, intraarterial, intrathecal, intracapsular, intraorbital, intracardiac, intradermal, transdermal, transtracheal, subcuticular, intraarticulare, subcapsular, subarachnoid, intraspinal and intrasternal, oral, sublingual buccal, rectal, vaginal, nasal ocular administrations, as well as infusion, inhalation, and nebulization. The phrases “parenteral administration” and “administered parenterally” as used herein means modes of administration other than enteral and topical administration.

### II. Regenerative Nonsteroidal Anti-Inflammatory (RNSA) Compositions

The present disclosure provides a cell-free or substantially cell-free regenerative nonsteroidal anti-inflammatory (RNSA) composition suitable for therapeutic or prophylactic use comprising an isolatable cell-free or substantially cell-free placenta-derived extract obtained from placental tissue from one or more mammalian donors, wherein said extract may comprise one or more eicosanoids optionally selected from 6kPGF1α, TXB2, PGF2α, PGE2, PGA2, LTB4, 5oxoETE, 5HETE, 11HETE, 12HETE, 15HETE, 20HETE, 5,6DHET, 8,9DHET, 11,12DHET, 14,15DHET, 9HODE, 13HODE, and AA. The composition may be capable of inhibiting proliferation of activated T cells and may further be non-cytotoxic for one or more cells selected from stromal cells, mesenchymal stromal cells (MSCs), parenchymal cells, and tenocytes in a subject, in vivo, or in vitro.

The cell-free or substantially cell-free regenerative nonsteroidal anti-inflammatory (RNSA) compositions derived from placenta provided herein may be specifically derived from placental “perinatal stromal cells” (PSCs), also referred to herein as mesenchymal stromal cells (MSCs) or which may comprise MSCs. More specifically, the cell-free or substantially cell-free regenerative nonsteroidal anti-inflammatory compositions may be derived from placental MSCs which have undergone apoptosis, e.g., naturally and/or by inducing apoptosis by exogenous means, optionally by the use of one or more of the methods disclosed or exemplified herein or other means known in the art for inducing cell apoptosis. It is also contemplated herein that the cell-free or substantially cell-free regenerative nonsteroidal anti-inflammatory (RNSA) compositions described herein may be derived from non-placental MSCs obtained from, e.g., bone marrow, adipose tissue, muscle, corneal stroma, and/or deciduous teeth dental pulp, i.e., “adult” MSCs.

As used herein, “perinatal stromal cell,” or “PSC” refers to cells isolated from a placenta. The placenta may be a human placenta or may be derived from any other mammal such as a non-human primate, a pig, a sheep, a horse, a cow, a dog, a cat, a rat, or a mouse. The placenta may preferably be a human placenta. The human placenta includes an umbilical cord, an amniotic membrane (amnion), and a “placenta proper”, which includes the chorion or chorionic plate, the villus, the intervillous space, the basal plate and the cotyledon. Each portion of the placenta can be isolated and can be used to derive subpopulations of perinatal stromal cells.

In some embodiments, the placental tissue may be obtained from a single donor. In some embodiments, the placental tissue may be obtained from more than one donor (pooled donor placental tissue sample).

In one embodiment, the placenta may comprise at least one placental tissue selected from amniotic membrane, chorion membrane, chorionic villus, umbilical cord, and Wharton's Jelly. In preferred embodiments, the placenta may comprise at least one placental tissue selected from amniotic membrane and/or chorion membrane.

The amnion membrane can be mechanically separated from the chorion, which leads to the derivation of amnion perinatal stromal cell (APSC). When sectioned longitudinally, the umbilical cord exposes Wharton's jelly, containing umbilical arteries and vein. After removal of the blood vessels, Wharton's Jelly perinatal stromal cell (WPSC, WJPSC, or MJ-MSC) can be derived from the umbilical cord. When the amnion and the umbilical cord are removed, the remaining portion of the placenta, which can be referred to as the placenta proper, can be used directly to prepare placenta proper stromal cell (PPSC), or can be further separated. For example, the chorionic membrane can be detached to isolate whole chorion derived stromal cell (CSC), and the intermediate and terminal villi can be exposed to isolate chorionic-villi stromal cell (CVC).

In some embodiments, the at least one placental tissue may comprise perinatal stromal cells (PSCs) and/or mesenchymal stromal cells (MSCs).

In certain embodiments, the RNSA composition may be stable in solution at room temperature for a prolonged time, e.g., at least 1, 2, 3, 4, 5, 6, 7 or at least 8 weeks or more.

In certain embodiments, the RNSA composition may be stable to lyophilization.

In some embodiments, the RNSA composition may elicit an anti-inflammatory response. As such, the RNSA composition may be capable of inhibiting proliferation of activated T cells in a subject, in vivo, or in vitro, wherein the T cells are CD4+, CD8+, CD4+/CD8+, CD11c+, CD11b+, and/or CD56+ T cells.

In some embodiments, the RNSA composition may be capable of promoting proliferation of one or more cells selected from stromal cells, mesenchymal stromal cells (MSCs), parenchymal cells, and tenocytes in a subject, in vivo, or in vitro.

Additionally, in some embodiments, the RNSA composition may be capable of reducing expression of one or more pro-inflammatory cytokines from activated peripheral blood mononucleated cells (PBMCs) and/or activated T cells and/or of promoting the expression or activity of one or more anti-inflammatory cytokines in a subject, in vivo, or in vitro. The one or more pro-inflammatory cytokines may be selected from TNFα, NFκB, IL17A, IL-6, and IFNγ.

And in some embodiments, the RNSA composition may be capable of increasing cAMP production from activated T cells in a subject, in vivo, or in vitro.

### III. Methods for Producing Regenerative Nonsteroidal Anti-Inflammatory Compositions

The present disclosure also generally encompasses a method for producing a cell-free or substantially cell-free regenerative nonsteroidal anti-inflammatory (RNSA) composition. The method may comprise (i) obtaining at least one placental tissue from a mammal selected from human, non-human primate, pig, sheep, horse, cow, dog, cat, rat, and mouse, wherein the at least one placental tissue is selected from amniotic membrane, chorion membrane, chorionic villus, umbilical cord, and Wharton's Jelly, and wherein the at least one placental tissue comprises perinatal stromal cells (PSCs); (ii) optionally isolating the PSCs from said placental tissue and culturing the PSCs in at least one cell culture medium (iii) permitting apoptosis of said placental tissue and PSCs comprised therein and/or permitting apoptosis of PSCs isolated therefrom to occur naturally and/or inducing or enhancing apoptosis of said placental tissue and PSCs and/or inducing apoptosis of PSCs isolated therefrom to produce an extract; and (iv) separating the extract or a portion thereof from the cells and tissue, for example, by decantation, centrifugation, and/or filtration; thereby producing the cell-free or substantially cell-free regenerative nonsteroidal anti-inflammatory composition and (v) optionally further purifying or concentrating said extract or the actives comprised therein, e.g., by concentrating the amount of one or more eicosanoids optionally selected from 6kPGF1α, TXB2, PGF2α, PGE2, PGA2, LTB4, 5oxoETE, 5HETE, 11HETE, 12HETE, 15HETE, 20HETE, 5,6DHET, 8,9DHET, 11,12DHET, 14,15DHET, 9HODE, 13HODE, and AA, in order to enhance its anti-inflammatory potency. In some embodiments, the PSCs may comprise MSCs. In some embodiments, the mammal may be a human.

In some embodiments, the method may further comprise conducting one or more screening assays to assess the effects of the isolated apoptotic extract or one or more portions thereof on the proliferation of activated T cells and/or the proliferation of one or more cells selected from stromal cells, mesenchymal stromal cells (MSCs), parenchymal cells, and tenocytes and/or on the expression of pro-inflammatory cytokines and/or the expression of anti-inflammatory cytokines in a mammalian subject, in vivo, or in vitro.

In some embodiments, different portions of the isolated apoptotic extract may be screened in order to assess potency.

In some embodiments, inducing apoptosis may comprise serum deprivation, nutrient deprivation, and/or hypoxia.

In exemplary embodiments, inducing apoptosis may comprise (i) contacting the placental tissue with a non-cell culture medium in a ratio ranging from about 1 mL non-cell culture medium per 1 gram of placental tissue to about 100 mL non-cell culture medium per 1 gram of placental tissue, preferably in a ratio of about 10 mL non-cell culture medium per 1 g of placental tissue; and (ii) incubating the placental tissue in the non-cell culture medium in an air-tight environment at a temperature ranging from about 4° C. to about 42° C., preferably at about 37° C., for about 2 days to about 12 days, preferably for about 10 days, wherein the incubating optionally comprises agitation, for example, at about 90 rpm. However, as afore-mentioned apoptosis may alternatively be induced or enhanced by other methods known in the art for initiating or promoting apoptosis.

In some embodiments, the method may further comprise washing the placental tissue with phosphate-buffered saline (PBS) prior to inducing apoptosis. In some embodiments, the method may further comprise mincing the placental tissue prior to inducing apoptosis. In some embodiments, the method may further comprise contacting the placental tissue with one or more antimicrobial agents.

In some embodiments, the non-cell culture medium may comprise saline solution. In some embodiments, the saline solution may comprise 0.9% NaCl. In some embodiments, the saline solution may comprise phosphate-buffered saline (PBS). In some embodiments, the air-tight environment may prevent gas exchange, thereby inducing a hypoxic environment.

In some aspects, the method may further comprise isolating the placental tissue PSCs and culturing the PSCs in at least one cell culture medium prior to inducing apoptosis. In some embodiments, inducing apoptosis may comprise (i) replacing the at least one cell culture medium with a non-cell culture medium; and (ii) incubating the cultured PSCs in the non-cell culture medium in an air-tight environment at a temperature ranging from about 4° C. to about 42° C., preferably at about 37° C., for about 3 days to about 5 days, preferably for about 4 days, wherein the incubating optionally comprises agitation. In some embodiments, the cultured PSCs may be cultured to at least 80% confluence.

In some embodiments, the non-cell culture medium may comprise saline solution. In some embodiments, the saline solution may comprise 0.9% NaCl. In some embodiments, the saline solution may comprise phosphate-buffered saline (PBS). In some embodiments, the air-tight environment may prevent gas exchange, thereby inducing a hypoxic environment.

In some embodiments, the method may further comprise washing the cultured PSCs with phosphate-buffered saline (PBS) prior to inducing apoptosis.

In some embodiments, the method may further comprise centrifugation at about 10,000×g for about 30 minutes.

In some embodiments, the method may further comprise filtration through a 0.45 membrane. In some embodiments, the method may further comprise filtration through a 0.2 μm membrane, i.e. sterile filtration. In some embodiments, the method may further comprise filtration through a 30 KDa MWCO membrane, a 10 KDa MWCO membrane, a 5 KDa MWCO membrane, a 3 KDa MWCO membrane, and/or a 2 KDa MWCO membrane.

The present disclosure also generally relates to a cell-free or substantially cell-free regenerative nonsteroidal anti-inflammatory (RNSA) composition produced by any of the methods disclosed herein.

### IV. Methods for Treating Inflammatory Diseases and Conditions

In a further embodiment, a method of treatment of at least one inflammatory condition or disease or at least one symptom associated therewith is provided. The method may comprise administering a therapeutically or prophylactically effective amount of the cell-free or substantially cell-free regenerative nonsteroidal anti-inflammatory (RNSA) composition described herein to a subject in need thereof wherein such treatment optionally may reduce or prevent tissue inflammation in the subject.

In one aspect, a molecular marker of inflammation in the tissue is decreased as compared to said molecular marker in the tissue before the administration of the RNSA composition. In some aspects, the molecular marker of inflammation is selected from the group consisting of TNFα expression, NFκB expression, INFγ expression, IL-17 (or IL-17A) expression, IL-6 expression, and a combination thereof.

In some embodiments, the at least one inflammatory condition or disease may be an acute or chronic condition associated with inflammation, e.g., an acute or chronic autoimmune disease associated with acute or chronic inflammation, optionally a viral or bacterial or fungal infection associated with acute or chronic inflammation, further optionally a hepatitis virus, ZIKA virus, herpes, papillomavirus, influenza virus, or coronavirus, further optionally COVID-19 or SARS. In some embodiments, the at least one inflammatory condition or disease may be an acute inflammatory condition or disease, e.g., an acute inflammatory autoimmune condition or infectious condition associated with acute inflammation such as a viral condition associated with acute inflammation, further optionally a coronavirus infection, e.g., COVID-19 or SARS.

In some embodiments, the at least one inflammatory condition or disease or symptom associated therewith may be selected from pneumonia, single or multiple organ failure or dysfunction, sepsis, cytokine storm, fever, neurological dysfunction or impairment, loss of taste or smell, cardiac dysfunction, pulmonary dysfunction, liver dysfunction, acute or chronic respiratory dysfunction, graft versus host disease (GVHD), cardiomyopathy, vasculitis, fibrosis, ophthalmic inflammation, dermatologic inflammation, gastrointestinal inflammation, tendinopathies, allergy, asthma, glomerulonephritis, pancreatitis, hepatitis, inflammatory arthritis, gout, multiple sclerosis, psoriasis, Acute Respiratory Distress Syndrome (ARDS), wound healing, diabetic ulcers, non-healing wounds, lupus, and at least one autoimmune disease associated with acute or chronic inflammation.

In some embodiments, the at least one inflammatory condition or disease may be pneumonia, e.g., caused by at least one virus, fungus, bacterium or a combination thereof. In exemplary embodiments, the pneumonia may be Covid-19-associated and/or influenza-associated pneumonia. Coronavirus Disease 2019 or Covid-19 is caused by severe acute respiratory syndrome coronavirus 2 (SARS-CoV-2 coronavirus). The predominant CT findings of Covid-19-associated pneumonia patients include conspicuous ground-glass opacification, consolidation, bilateral involvement, and peripheral and diffuse distribution.

In some embodiments, the at least one inflammatory condition or disease may comprise COVID-19 or other inflammatory condition or infection and the treatment or prevention may further comprise the administration of at least one other active, e.g., an anti-inflammatory agent such as an anti-inflammatory antibody or anti-inflammatory fusion protein, e.g., Embrel (etanercept), Humira (adalimumab), or an IL-6 antagonist, an antiviral agent, an antibacterial agent, an antifungal agent, an analgesic, an anti-congestive agent, an anti-fever agent, or a combination of any of the foregoing.

In some embodiments, the treated subject has been diagnosed with or is suspected of having a coronavirus infection, optionally COVID-19.

In some embodiments, the treated subject has been diagnosed with a coronavirus infection, optionally COVID-19, and is on a respirator, has Acute Respiratory Distress Syndrome (ARDS), and/or is experiencing respiratory difficulties.

In some embodiments, the treated subject has been diagnosed with or suspected of having a coronavirus infection, optionally COVID-19, and the subject comprises one or more risk factors that place the subject at higher risk for morbidity or a poor treatment outcome, e.g., age over 55 years, obesity, diabetes, cardiac problem or condition, respiratory condition, optionally asthma, COPD, cystic fibrosis, is a smoker, is a heavy drinker, has lupus, has elevated blood pressure, has cancer, receives chemotherapy, has (chronic) kidney disease and/or is on dialysis, or any combination of the foregoing.

In some embodiments, the ophthalmic inflammation may comprise one or more of corneal regeneration, corneal wound healing, corneal melting, dry eye, ocular infection, eyelid sty, and autoimmune-associated peripheral ulcerative keratitis.

In some embodiments, the at least one inflammatory condition or disease may be fibrosis. In exemplary embodiments, the fibrosis may comprise pulmonary fibrosis, cystic fibrosis, idiopathic pulmonary fibrosis, interstitial pulmonary fibrosis, radiation-induced lung injury, liver fibrosis, bridging fibrosis of the liver, cirrhosis, glial scar, arterial stiffness, arthrofibrosis, Crohn's disease, Dupuytren's contracture, keloid fibrosis, Mediastinal fibrosis, Myelofibrosis, Myocardial fibrosis, Peyronie's disease, Nephrogenic systemic fibrosis, Progressive massive fibrosis, pneumoconiosis, Retroperitoneal fibrosis, stromal fibrosis, Scleroderma, systemic sclerosis, chronic obstructive pulmonary disease (COPD), asthma, and adhesive capsulitis.

In some embodiments, the at least one inflammatory condition or disease may be gastrointestinal inflammation. In exemplary embodiments, the gastrointestinal inflammation may comprise inflammatory bowel disease (IBD), Crohn's disease, ulcerative colitis, irritable bowel syndrome (IBS), and Celiac disease.

In some embodiments, the at least one inflammatory condition or disease may be ophthalmic inflammation. In exemplary embodiments, the ophthalmic inflammation may be associated with keratoconjunctivitis sicca.

In some embodiments, the at least one inflammatory condition or disease may be dermatologic inflammation. In exemplary embodiments, the dermatologic inflammation may be selected from eczema and psoriasis.

In some embodiments, the at least one inflammatory condition or disease may be at least one autoimmune disease selected from Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome, (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndrome type I, Polyglandular syndrome type II, Polyglandular syndrome type III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, and Vogt-Koyanagi-Harada Disease.

In exemplary embodiments, a therapeutically or prophylactically effective amount of the cell-free or substantially cell-free regenerative nonsteroidal anti-inflammatory (RNSA) composition may be administered to a subject in need thereof. The therapeutically effective amount may comprise one or more doses of the composition. Each dose may range from 0.1 mL/10 kg body weight to 10 mL/10 kg body weight. In some preferred embodiments, the dose may be 1 mL/10 kg body weight.

In some embodiments, the composition may be administered by one or more of injection, optionally intravenous (IV) or subcutaneous (SC) administration, nebulization, and/or eye drops.

In some embodiments, the subject may be selected from a human, non-human primate, pig, sheep, horse, cow, dog, cat, rat, and mouse. In preferred embodiments, the subject may be human.

In some embodiments, the method of treatment or prevention may further comprise the administration of at least one other active, e.g., an anti-inflammatory agent such as an anti-inflammatory antibody or anti-inflammatory fusion protein, an antiviral agent, an antibacterial agent, an antifungal agent, an analgesic, an anti-congestive agent, an anti-fever agent, or a combination of any of the foregoing.

In exemplary embodiments, the subject may have been diagnosed with or is suspected of having a coronavirus infection, optionally COVID-19.

In some embodiments, the subject may have been diagnosed with a coronavirus infection, optionally COVID-19, and is on a respirator, has Acute Respiratory Distress Syndrome, and/or is experiencing respiratory difficulties.

In some embodiments, the subject may have been diagnosed with or suspected of having a coronavirus infection, optionally COVID-19, and optionally the subject comprises one or more risk factors that place the subject at higher risk for morbidity or a poor treatment outcome, e.g., age over 55 years, obesity, diabetes, cardiac problem or condition, respiratory condition, optionally asthma, COPD, cystic fibrosis, is a smoker, is a heavy drinker, has lupus, has elevated blood pressure, has cancer, receives chemotherapy, has (chronic) kidney disease and/or is on dialysis, or any combination of the foregoing.

All of the compositions and/or methods disclosed and claimed herein can be made and executed without undue experimentation in light of the present disclosure. While the compositions and methods have been described in terms of preferred embodiments, it will be apparent to those of skill in the art that variations and substitutions may be applied to the compositions and/or methods and in the steps or in the sequence of steps of the methods described herein without departing from the concept, spirit and scope of the disclosure.

### V. Examples

The following examples are provided for illustrative purposes only and are non-limiting.

### Example 1: Generation and Isolation of Regenerative Nonsteroidal Anti-Inflammatory (RNSA) Compositions from Placental Tissue

Human placentas were collected by selective C-section after maternal consent and according to the guidelines of the ethical committee of the Cooperative Human Tissue Network at the University of Alabama. Human placental tissues were processed within 24 hours of collection in a sterile laminar hood as follows.

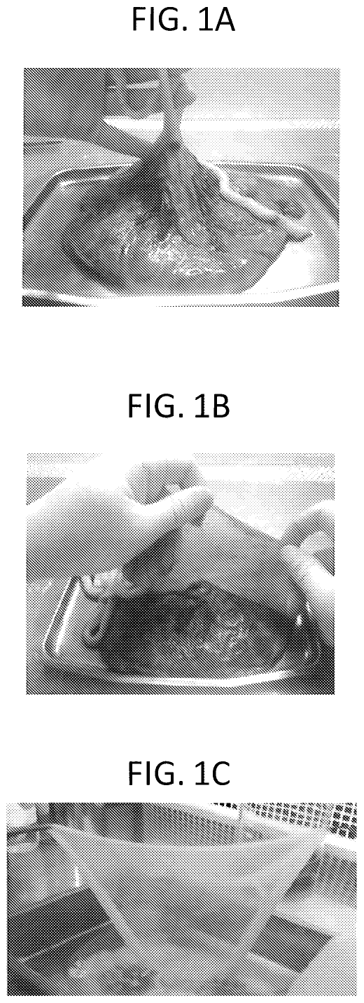

In a laminar flow hood, the amniotic membrane was mechanically separated from the chorion and umbilical cord and subsequently washed extensively with phosphate-buffered saline (PBS) with 1% Primocin™ (Invivogen) (FIG. 1A-C). The amniotic membrane was separated in an Erlenmeyer flask.

The chorion membrane, chorionic villus, and umbilical cord (Wharton's Jelly) were each treated similarly as the amniotic membrane.

The amniotic membrane (or chorion membrane, or chorionic villus, or Wharton's Jelly/umbilical cord tissue) was incubated in a 0.9% NaCl saline solution in an air-tight environment (a capped Erlenmeyer flask) at 37° C. for 10 days with gentle agitation at 90 rpm to induce apoptosis of amniotic membrane cells (i.e., amniotic membrane-derived mesenchymal stromal cells). The supernatant was decanted from the placental tissue and was centrifuged at 10,000×g for 30 min. The supernatant was then filtered through a 0.45 μm membrane and then through a 0.22 μm membrane (VWR) or directly through a 0.22 μm membrane (Pall) to obtain the cell-free or substantially cell-free regenerative nonsteroidal anti-inflammatory composition (“RNSA”). The RNSA composition was stored at −80° C. until use.

### Example 2: Generation and Isolation of Regenerative Nonsteroidal Anti-Inflammatory (RNSA) Compositions from Cultured Placental Mesenchymal Stromal Cells

Human perinatal stromal cells ((hPSCs) which comprise human mesenchymal stromal cells (hMSCs)) were obtained from the placentas described in Example 1 as follows.

The amnion membrane was mechanically separated from the chorion and washed extensively with phosphate-buffered saline (PBS). It was then minced into small pieces and digested with TrypLE (Gibco, Waltham, Mass., USA) at 5 mL/g of tissue for 30 min in a shaker incubator (124 Incubator Shaker series, New Brunswick Scientific, Edison, N.J., USA) at 37° C., 150 rpm to remove the amniotic epithelial cells. The undigested amnion was then removed, washed with PBS and further digested with 125 U/mg Collagenase I (Worthington, Lakewood, N.J., USA) at 37° C., 150 rpm for 1.5 h to isolate the amniotic mesenchymal cells (APSC). The mobilized cells in the digest were passed through a 100 μm cell strainer (VWR, Radnor, Pa., USA) and collected by centrifugation at 500×g for 8 min.

The Wharton's Jelly (WPSC) was extracted from the umbilical cord as follows: the umbilical cord was sectioned in approximately 1.5 cm in length pieces and then dissected longitudinally to expose the Wharton's Jelly. The arteries and vein were removed, the remaining tissue was minced into small pieces, and digested with 125 U/mg Collagenase I at 37° C., 150 rpm for 2.5 h or until all tissue was digested. The digest was passed through a 100 μm cell strainer and centrifuged at 500×g for 8 min.

The chorion was mechanically separated from the amnion membrane and washed extensively with phosphate-buffered saline (PBS). The chorion membrane was then minced into small pieces and digested with 125 U/mg Collagenase I (Worthington, Lakewood, N.J., USA) at 37° C., 150 rpm for 1.5 h in a shaker incubator (124 Incubator Shaker series, New Brunswick Scientific, Edison, N.J., USA) to isolate the chorion stromal cells (CSCs). The mobilized cells in the digest were passed through a 100 μm cell strainer (VWR, Radnor, Pa., USA) and collected by centrifugation at 500×g for 8 min. Finally, the placental proper tissue was carefully removed to expose the intermediate and terminal villi, dissected at the base of the intermediate villi and thoroughly washed with PBS, minced into small pieces, and digested with 125 U/mg Collagenase I at 37° C., 150 rpm for 1.5 h to isolate the chorionic villi stromal cells (VSCs). The digest was passed through a 100 μm cell strainer and centrifuged at 500×g for 8 min to collect the mobilized perinatal stromal cells (PSCs).

Perinatal Stromal Cells (PSCs) isolated herein had similar characteristics of Mesenchymal Stromal Cells (MSCs) based on the ISCT criteria, which is being plastic adherent under standard culture conditions, expression of CD105+, CD73+, CD90+, CD11b−, and CD45− HLADR. However, the capability of PSCs to differentiate into tri-lineage (Chondrocyte, Osteocyte and Adipocyte) was not evaluated, since it is not suspected that the effect of the compositions derived from PSCs is based on their differentiation capabilities (sternness). Hence, PSCs are referred to herein as MSCs. In addition, some other immune-pertinent markers were tested to further identify such cells. All PSCs were positive (>70%) for CD273+ (PD-L2), CD210+ (IL-10 Receptor) and negative (<5%) for CD178− (FasL), CD119− (IFNg Receptor), CD85d− (ILT4) and CD40. These additional immune-regulatory markers could be used to extend the characterization panel to identify such cells.

The collected hMSCs derived from the amniotic membrane, Wharton's Jelly, chorion membrane and chorionic villus were each cultured using standard procedures known in the art. For each of the above types of placental hMSCs, 10.5 million cultured hMSCs were expanded in a PBS Biotech MINI Bioreactor (RoosterBio) utilizing Synthemax™ II Microcarrier beads (Corning), which the cells adhere to, and 450 mL Rooster Media (Rooster Basal Media supplemented with Rooster Media Booster (RoosterBio)) with agitation at 25 rpm. Daily samples were taken to determine the number of cells per bead by fluorescence microscopy with DAPI staining. On Day 3, 10 mL Rooster Replenish were added to the cells and agitation increased to 30 rpm. Cells were harvested on Day 5 after reaching at least 80% confluence. The culture media was removed and the cells and beads were washed in 300 mL CTS″ Dulbecco's phosphate-buffered saline (PBS) without calcium chloride, without magnesium chloride (ThermoFisher). About 250-300 mL of TrypLE Express (ThermoFisher) were added and incubated for 15 minutes without agitation and for 20 to 40 minutes with agitation at 40 rpm to dispatch cells from the beads. Cells were separated from the beads by filtration and centrifugation.

The cultured and expanded hMSCs were then incubated in 500 mL 0.9% NaCl saline solution in an air-tight environment at 37° C. for about 4 days with gentle agitation (30 rpm) to induce apoptosis. After about 4 days, greater than 95% of the cells will have undergone apoptosis as determined using fluorescence microscopy and staining with the LIVE/DEAD™ Viability/Cytotoxicity Kit (ThermoFisher). The liquid was then decanted and filtered through a 0.22 μm membrane to obtain the cell-free regenerative nonsteroidal anti-inflammatory composition (“RNSA”). The RNSA composition was stored at −80° C. until use.

### Example 3: Characterization of Cell-Free Regenerative Nonsteroidal Anti-inflammatory (RNSA) Compositions

### Activated Lymphocytes as a Model for Inflammation

Peripheral blood mononucleated cells (PBMCs) were isolated from whole human blood using Lymphoprep™ (STEMCELL™ Technologies) according to the manufacturer's protocol. T cells were isolated from the PBMCs using EasySep™ Release Human CD3 Positive Selection Kit (STEMCELL™ Technologies) according to the manufacturer's protocol. The T cells were then activated and expanded using ImmnoCult™ CD3/CD28/CD2 T cell activator (STEMCELL™ Technologies) according to the manufacturer's protocol. The activated T cell samples are referred to herein as “Tc3+Act” whereas the T cell samples which were not activated are referred to herein as “Tc3-Act.”

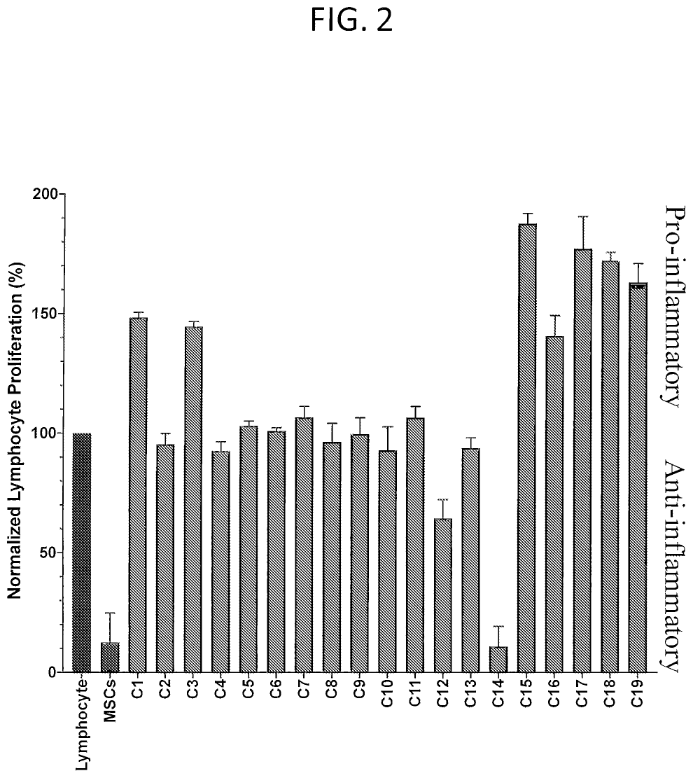

The activated and expanded T lymphocytes described above are a model for inflammation in which the normalized T cell proliferation represents 100% (FIG. 2). When the activated T cells are cultured in the presence of MSCs, inhibition of T cell proliferation of greater than 85% is observed (FIG. 2), representing a positive control for the inhibition of inflammation. Numerous tissues and conditions for treating those tissues were explored to determine a cell-free regenerative anti-inflammatory composition, including the amniotic membrane tissue and apoptotic condition described in Example 1 to produce the cell-free RNSA, shown in FIG. 2 as “C14.” The results for eighteen other conditions in which a cell-free composition was prepared and tested for its ability to inhibit activated T cell proliferation are also shown in FIG. 2. Of these nineteen conditions, only the cell-free regenerative anti-inflammatory composition derived from amniotic membrane tissue described in Example 1 inhibited activated T cell proliferation with a potency equal to or greater than MSCs (FIG. 2). Most conditions tested had no effect, whereas several conditions tested produced pro-inflammatory extracts (FIG. 2).

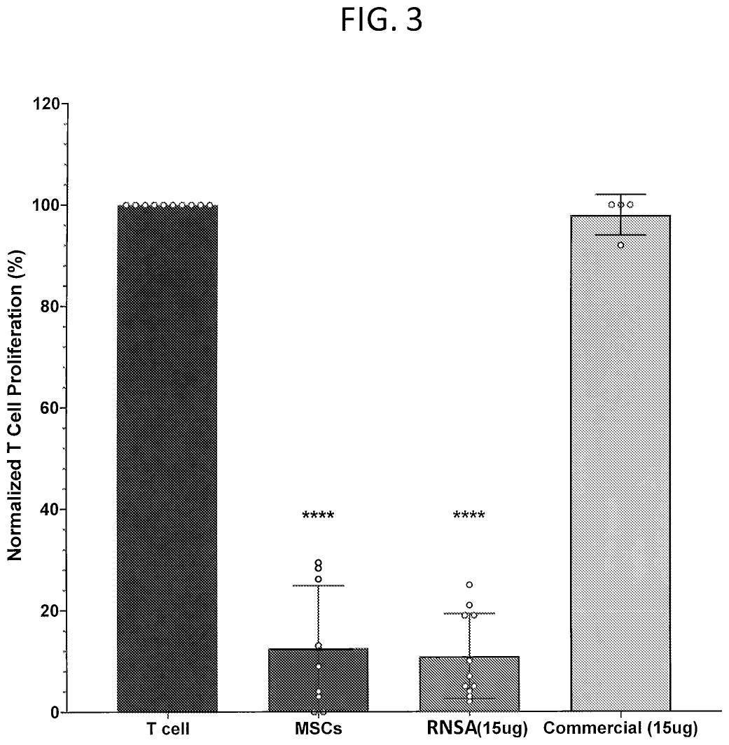

As described above, the activated and expanded T lymphocytes are a model for inflammation in which the normalized T cell proliferation represents 100% (FIG. 3). When the activated T cells are cultured in the presence of MSCs, inhibition of T cell proliferation of greater than 85% is observed (FIG. 3), representing a positive control for the inhibition of inflammation. Alternatively, when the activated T cells are cultured in the presence of 15 μg of the cell-free regenerative nonsteroidal anti-inflammatory composition (“RNSA”) derived from aminiotic membrane tissue described in Example 1, inhibition of T cell proliferation of greater than 85% is also observed (FIG. 3). In contrast, when the activated T cells are cultured in the presence of 15 μg of a commercial cell-free reagent (“commercial”), no inhibition of T cell proliferation was observed (FIG. 3).

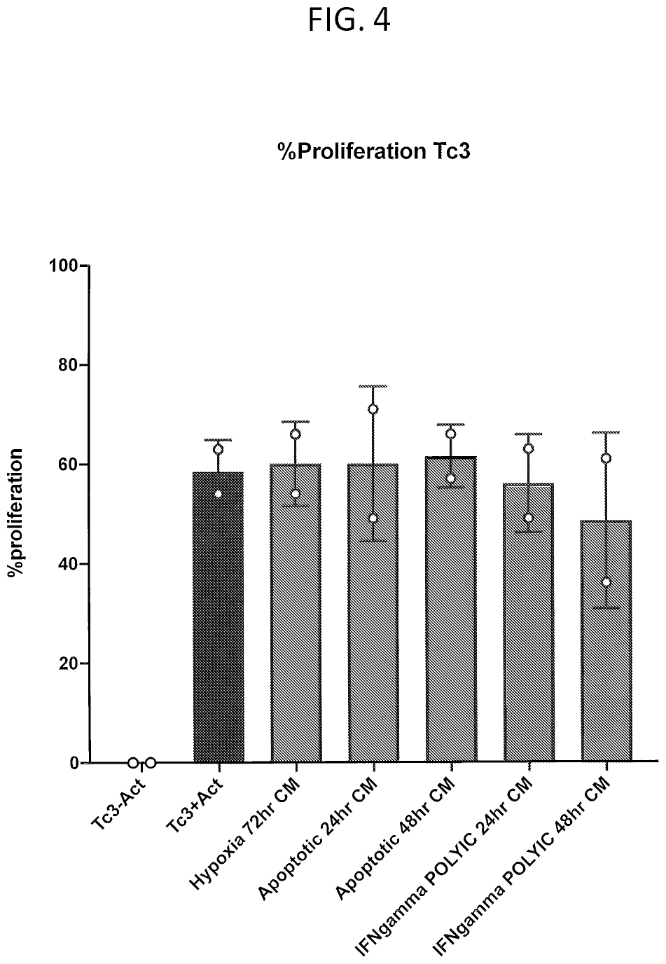

To determine whether the method of cell death has an effect on the potency of the RNSA composition, the protocol for generating the RNSA from amniotic membrane described in Example 1 was altered such that various means of cell death were tested. The amniotic tissue was subjected to a hypoxia condition for 72 hours, an apoptotic condition for 24 hours or 48 hours, or to IFN gamma and Poly(I:C) dsRNA for 24 hours or 48 hours. As shown in FIG. 4, none of these conditions produced an RNSA composition which inhibited activated T cell proliferation. This is in contrast to the amniotic membrane RNSA composition described in Example 1, which was produced using an apoptotic condition for 10 hours (FIG. 2 and FIG. 3).

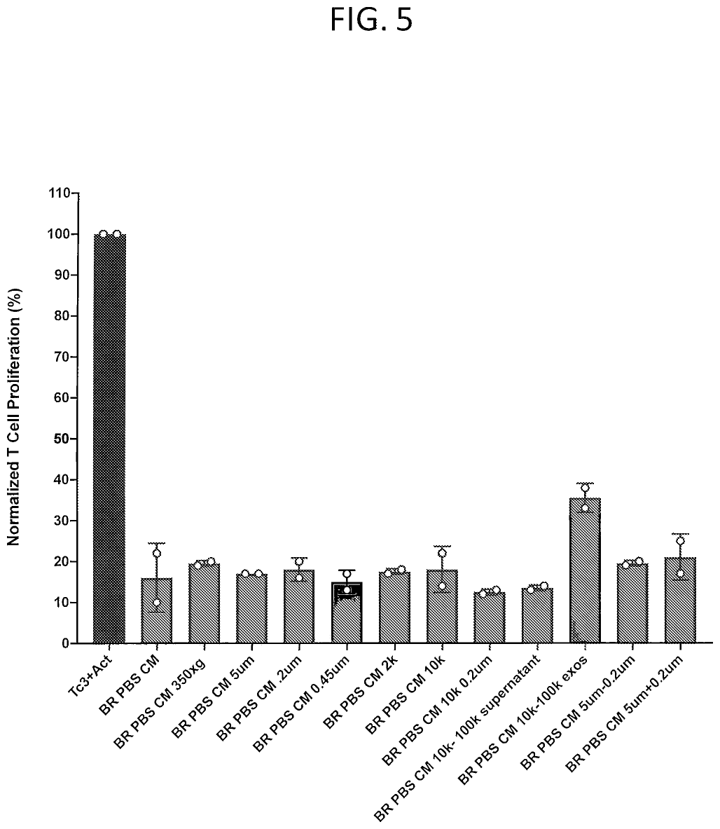

The T cell proliferation assay was also used to determine the effect of filtration on the potency of the RNSA composition. The RNSA composition was centrifuged at 350×g or filtered through membranes of various size cutoffs, including 5 μm, 0.45 μm, 0.2 μm, 2 KDa MWCO and 10 KDa MWCO, as shown in FIG. 5. Also tested were a 10 KDa-100 KDa filtration supernatant, and isolated exosomes “exos” from the supernatant, also shown in FIG. 5. The RNSA composition produced from amniotic membrane-derived hMSCs expanded in the PBS bioreactor (“BR PBS”) described in Example 2 was used in these tests. Condition media (“CM”) indicates the apoptotic condition of Examples 1 and 2. These results indicate that the bioactive molecule(s) of the RNSA composition is/are small molecule(s) less than 2 KDa, and that exosomes alone inhibited activated T cell proliferation less than the non-exosome fractions.

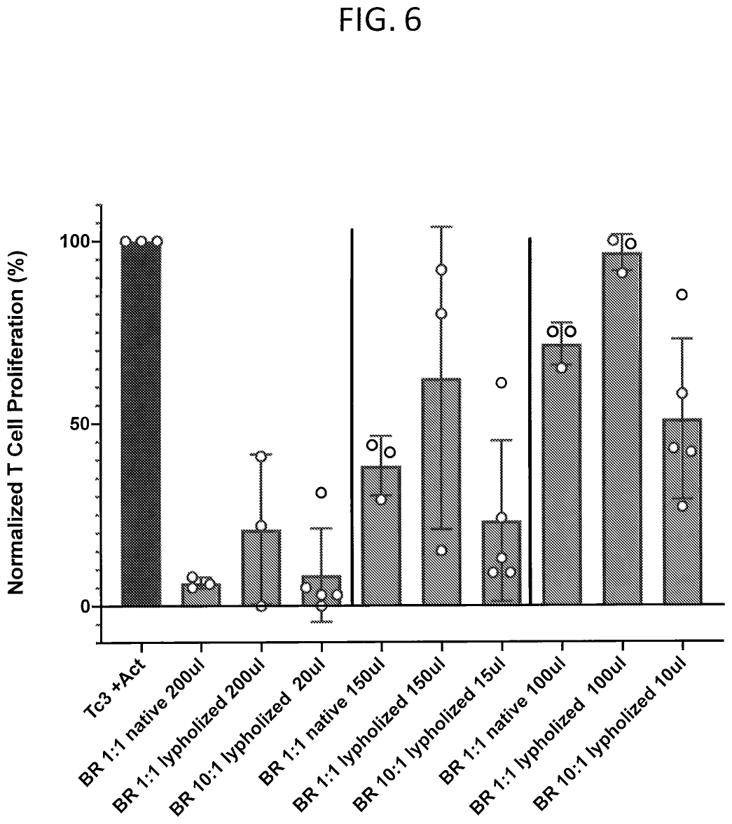

The T cell proliferation assay was also used to determine the effect of lyophilization on the potency of the RNSA composition. Here, the RNSA composition produced using cultured amnion hMSCs from Example 2 was used. Native RNSA samples of 200 μL, 150 μL, and 100 μL were compared to the same volume of native RNSA samples which were lyophilized and then resuspended in the equivalent volume of deionized water or 10-fold lower volume of deionized water. As shown in FIG. 6, lyophilization did not inhibit the bioactivity of the RNSA composition.

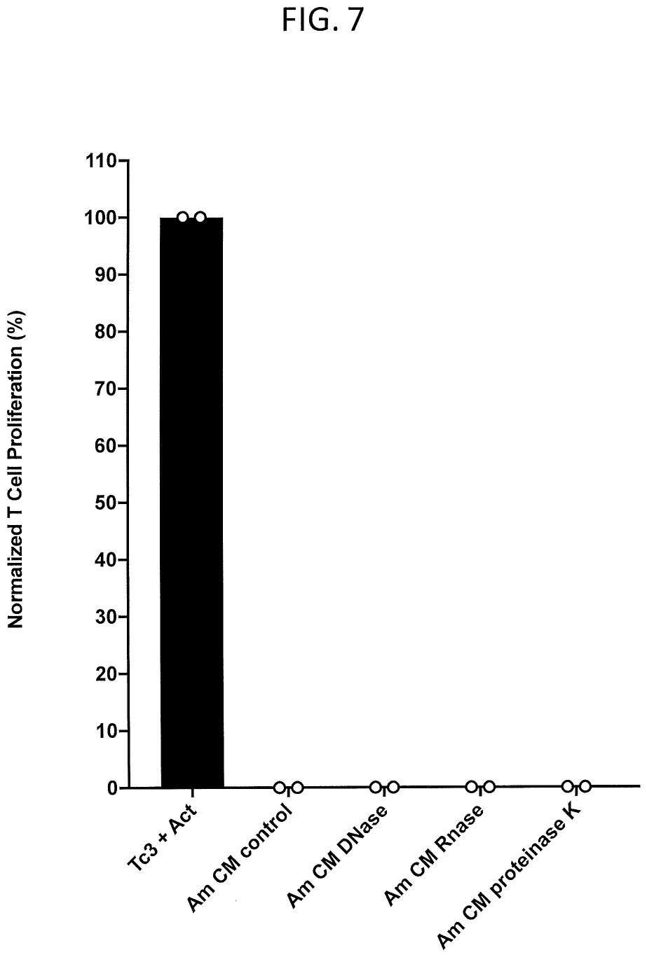

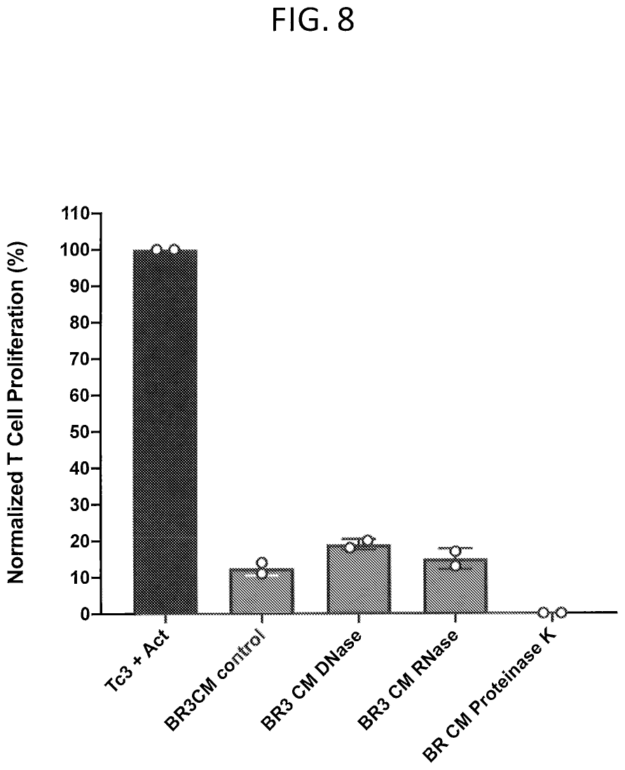

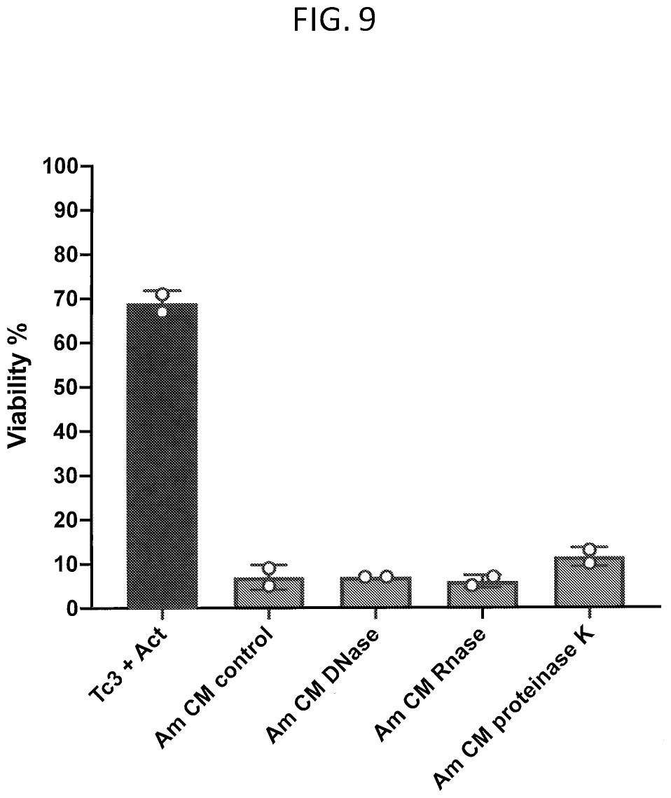

The T cell proliferation assay was also used to determine the effect of proteases, DNase, and RNase on the potency of the RNSA composition. As shown in FIG. 7, the potency of the RNSA composition produced from amniotic membrane tissue extraction described in Example 1 was not affected by DNase, RNase, or Proteinase K, suggesting a lipid nature for the bioactive compound(s). Similarly, as shown in FIG. 8, the potency of the RNSA composition produced from cultured hMSCs described in Example 2 was not affected by DNase, RNase, or Proteinase K. And as shown in FIG. 9, a similar T cell viability assay was used to determine that Proteinase K, DNase, and RNase do not affect the potency of the RNSA composition produced from amniotic membrane tissue extraction described in Example 1.

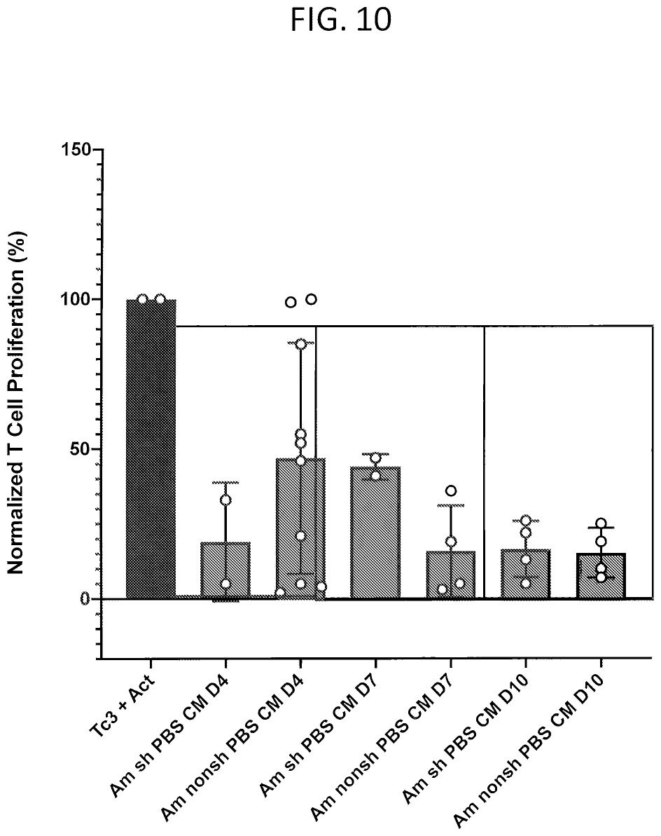

The T cell proliferation assay was also used to determine the effect of time during extraction and shaking vs. non-shaking conditions on the potency of the RNSA composition produced from amniotic membrane tissue extraction described in Example 1. As shown in FIG. 10, better extraction uniformity was achieved when waiting until Day 10. Also shown in FIG. 10, there was not a big effect from shaking vs. non-shaking when waiting until Day 10.

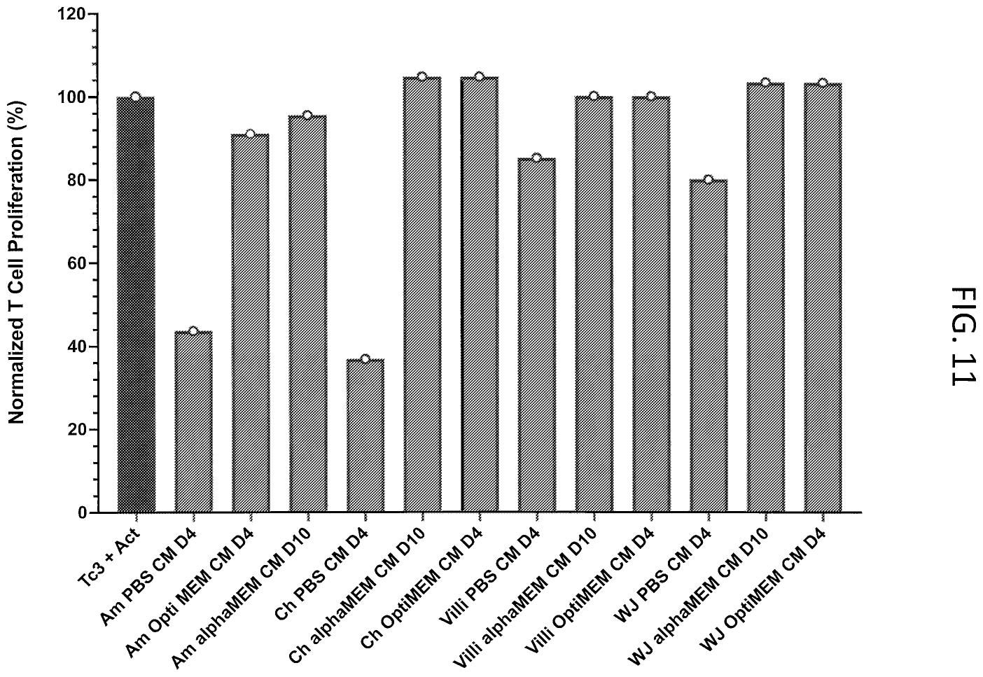

The T cell proliferation assay was also used to determine the effect of tissue type (AM—amniotic membrane, CH—chorion membrane, Villi—chorionic villus, WJ—Wharton's Jelly/Umbilical cord tissue) on the potency of the RNSA composition produced from the placental tissue extractions described in Example 1. The effect of the culture media (AlphaMEM, OptiMEM, and PBS) was also tested. As shown in FIG. 11, the RNSA compositions produced from amniotic membrane tissue extraction in PBS and from chorion membrane tissue extraction in PBS had the highest potency.

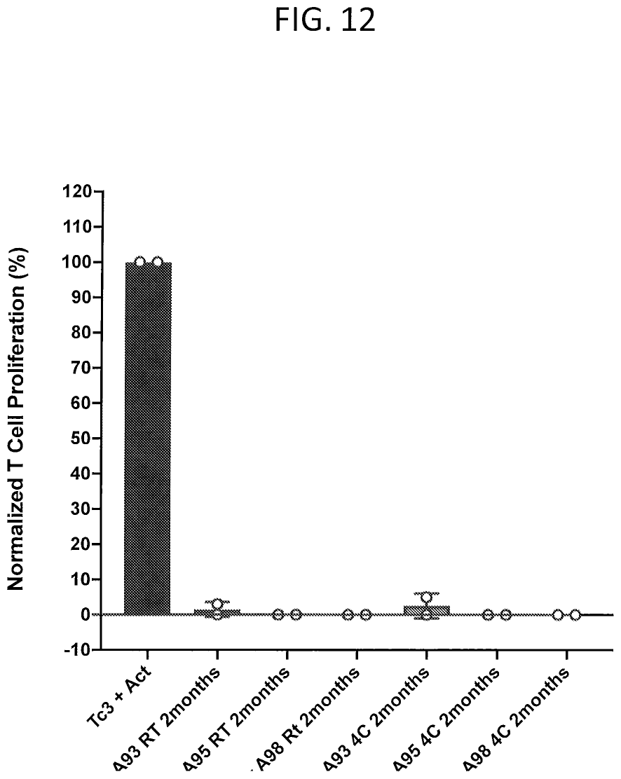

The T cell proliferation assay was also used to determine the stability of the RNSA composition at room temperature and 4° C. As shown in FIG. 12, various RNSA compositions (designated A93, A95, and A98) maintained potency for 2 months (greater than 8 weeks) at both room temperature and 4° C.

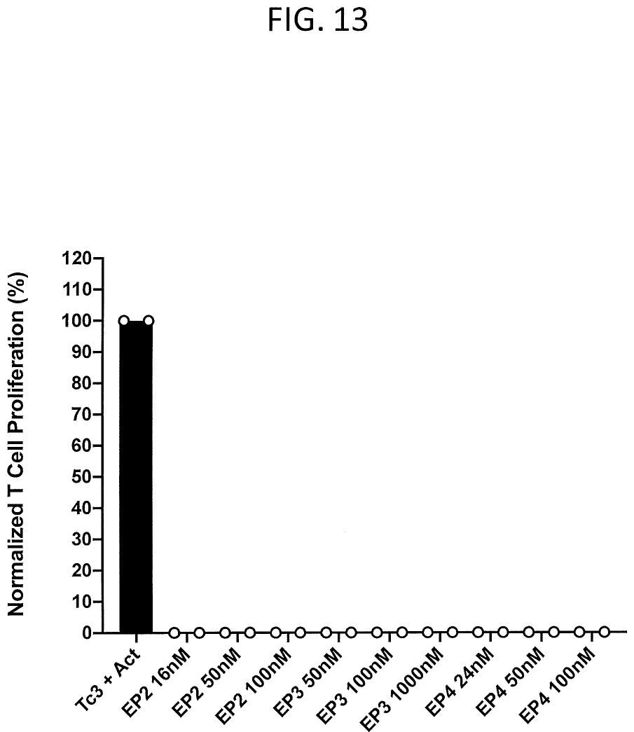

The T cell proliferation assay was also used to determine the effect of various concentrations of the EP2, EP3, and EP4 receptor blockers on the potency of the RNSA composition. As shown in FIG. 13, the EP receptor blockers did not affect the potency of the RNSA composition.

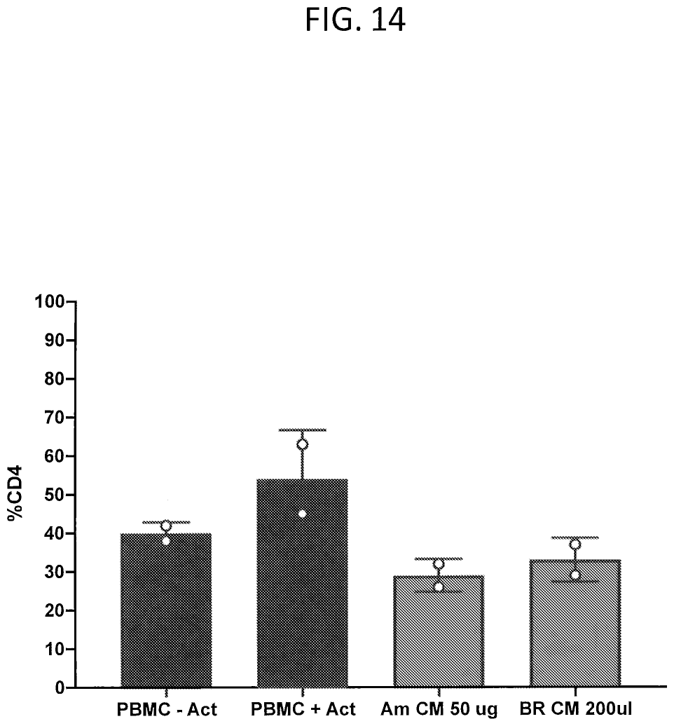

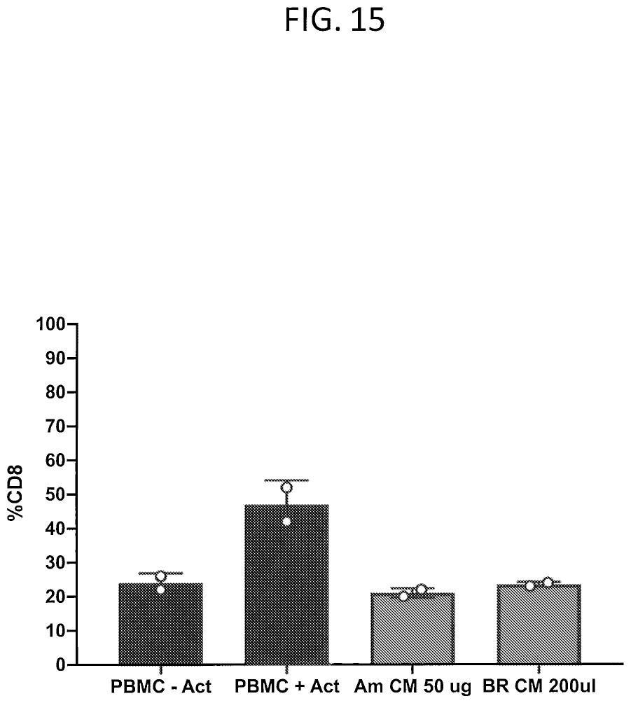

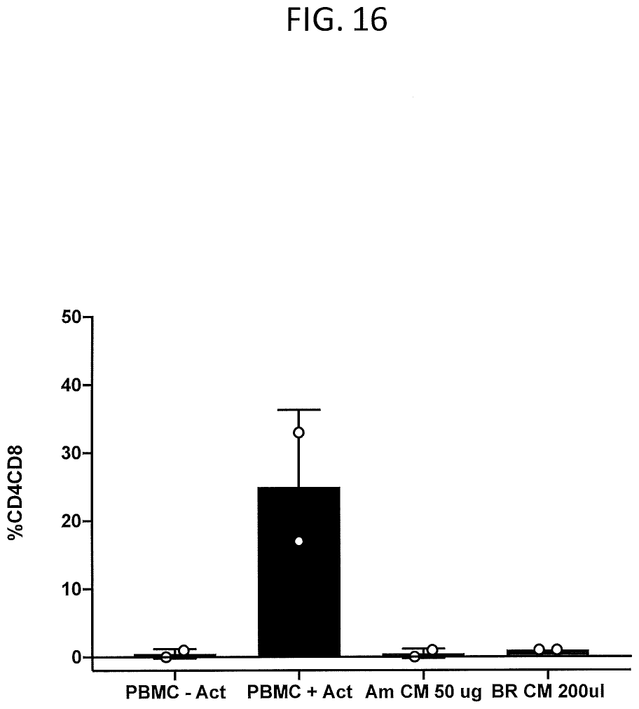

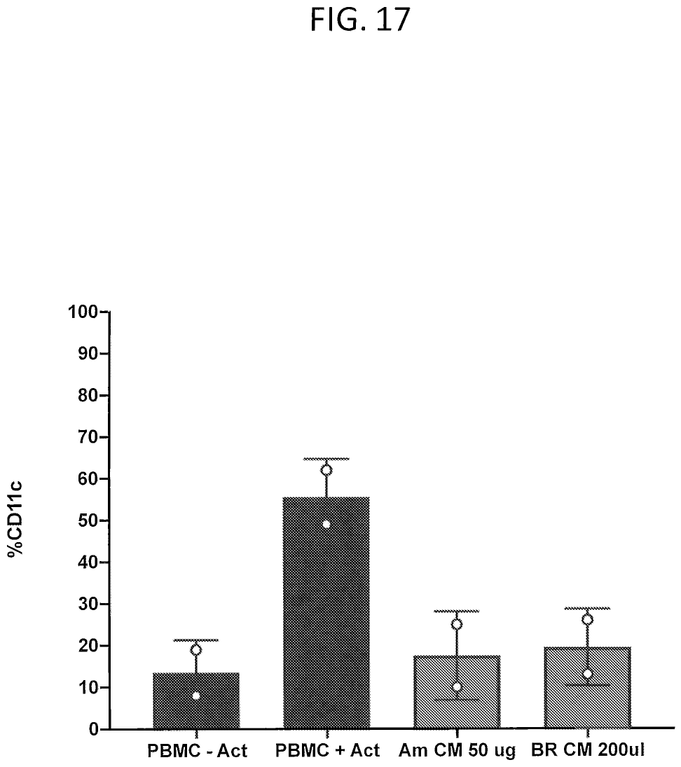

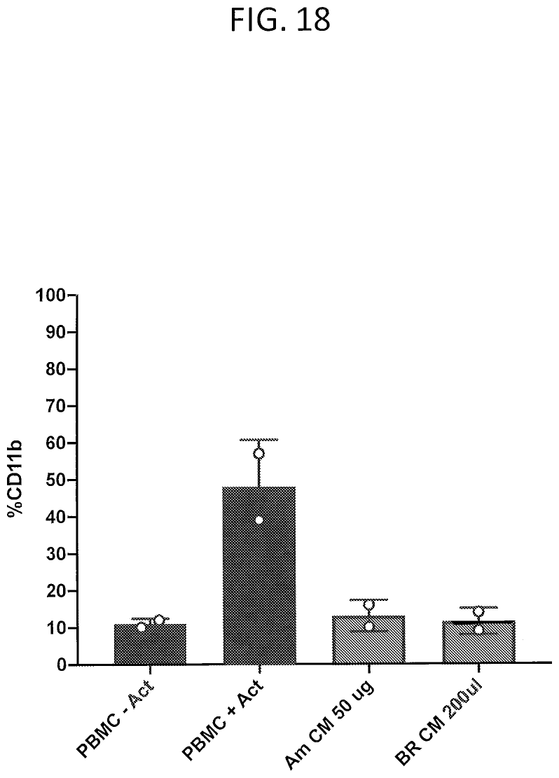

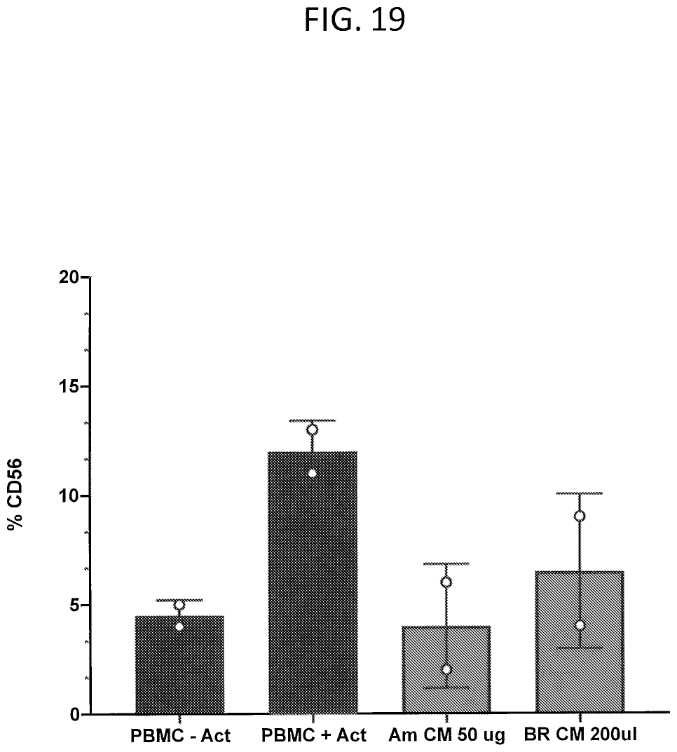

In some instances, a modified version of the T cell proliferation assay was utilized in which T cells were not isolated from the PBMCs. Rather, PBMCs were activated with the CD3/CD28/CD2 T cell activator and proliferation of CD4+ T cells was monitored. This modified proliferation assay was used to determine the effect of the tissue extraction process described in Example 1 (“AM”) compared to the cultured MSCs extraction process described in Example 2 (“BR”), as shown in FIG. 14, on the potency of the RNSA compositions. FIG. 15 shows the same modified T cell proliferation assay except that proliferation of CD8+ T cells was monitored. FIG. 16 shows the same modified T cell proliferation assay except that proliferation of CD4+/CD8+ T cells was monitored. FIG. 17 shows the same modified T cell proliferation assay except that proliferation of CD11c+ T cells was monitored. FIG. 18 shows the same modified T cell proliferation assay except that proliferation of CD11b+ T cells was monitored. FIG. 19 shows the same modified T cell proliferation assay except that proliferation of CD56+ T cells was monitored.

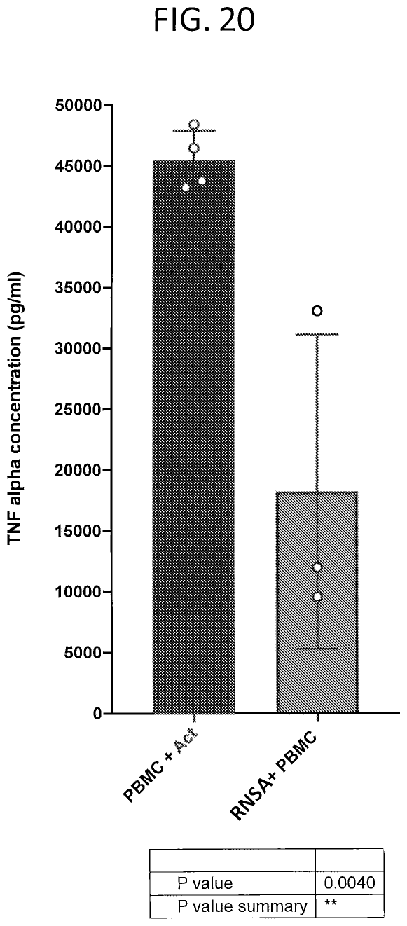

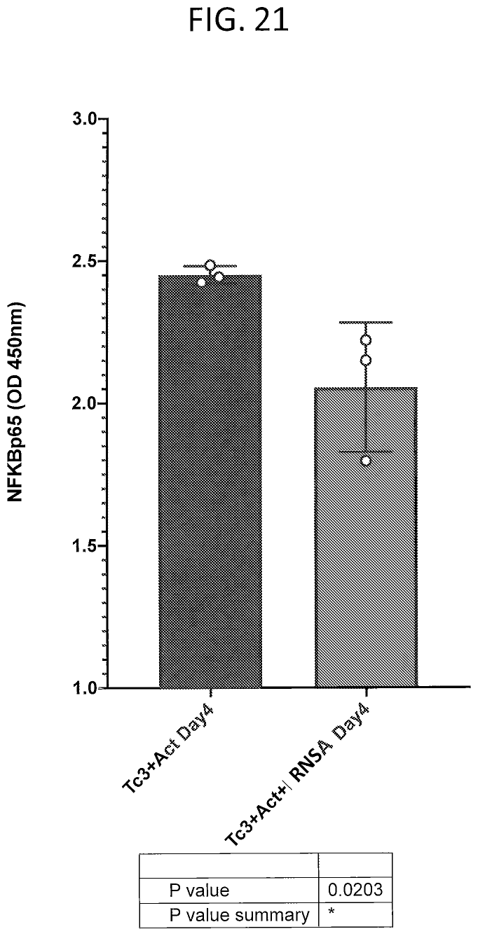

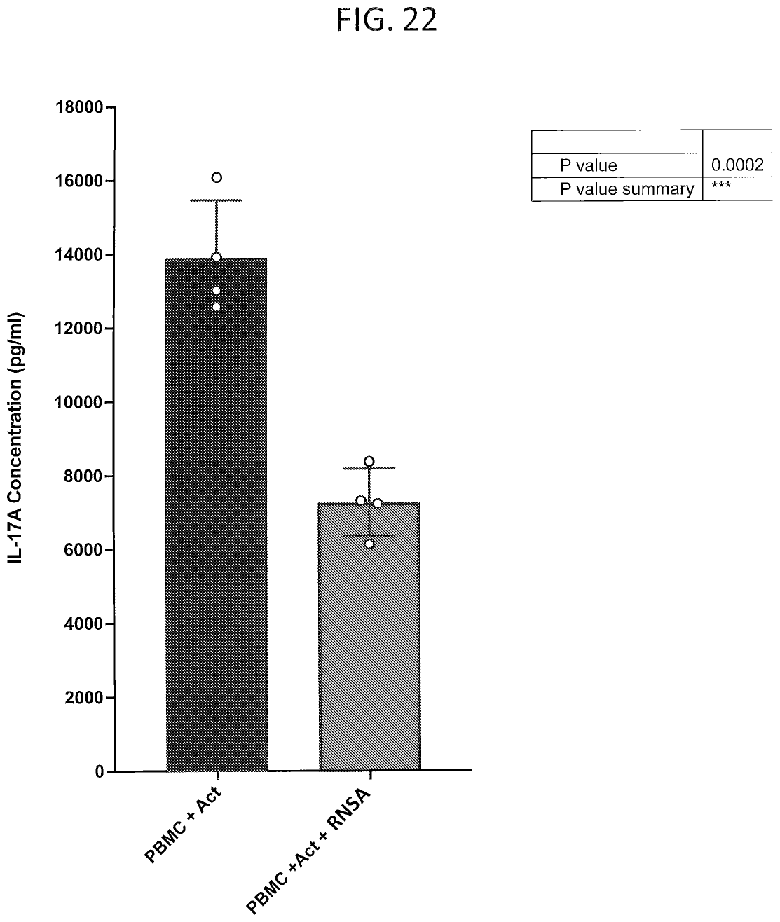

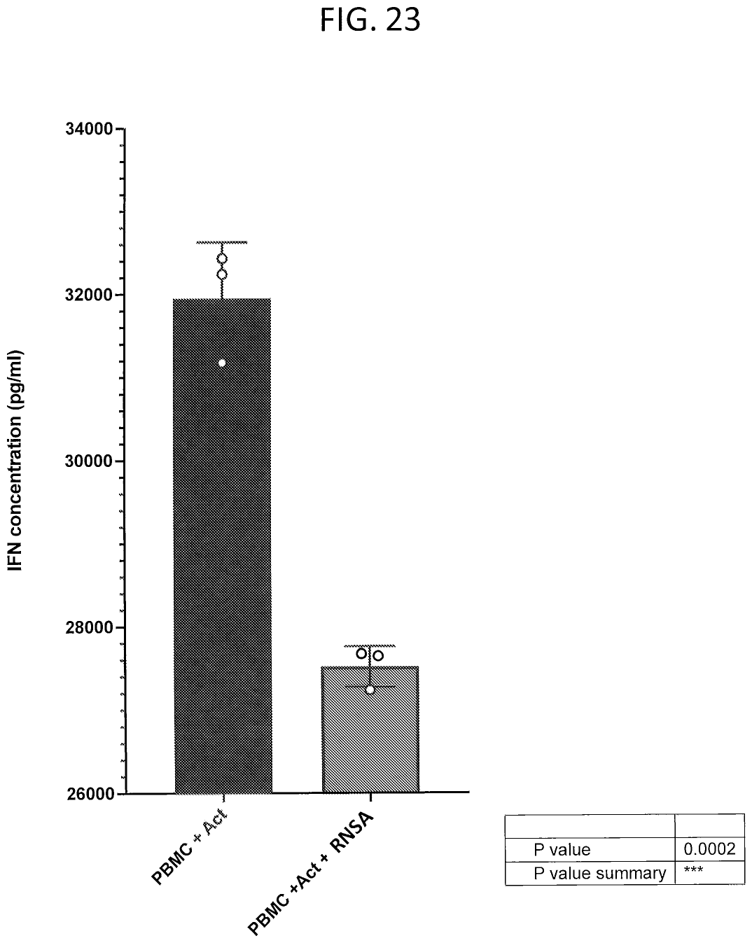

Next, the effect of the RNSA composition on the expression of pro-inflammatory cytokines was determined. Briefly, the amounts of pro-inflammatory cytokines were quantified in activated PBMCs in the absence and presence of the RNSA composition. FIG. 20 shows that the RNSA composition reduces the expression level of TNFα from activated PBMCs. FIG. 21 shows that the RNSA composition reduces the expression level of NFκB from activated PBMCs. FIG. 22 shows that the RNSA composition reduces the expression level of IL-17A from activated PBMCs. FIG. 23 shows that the RNSA composition reduces the expression level of IFNγ from activated PBMCs.

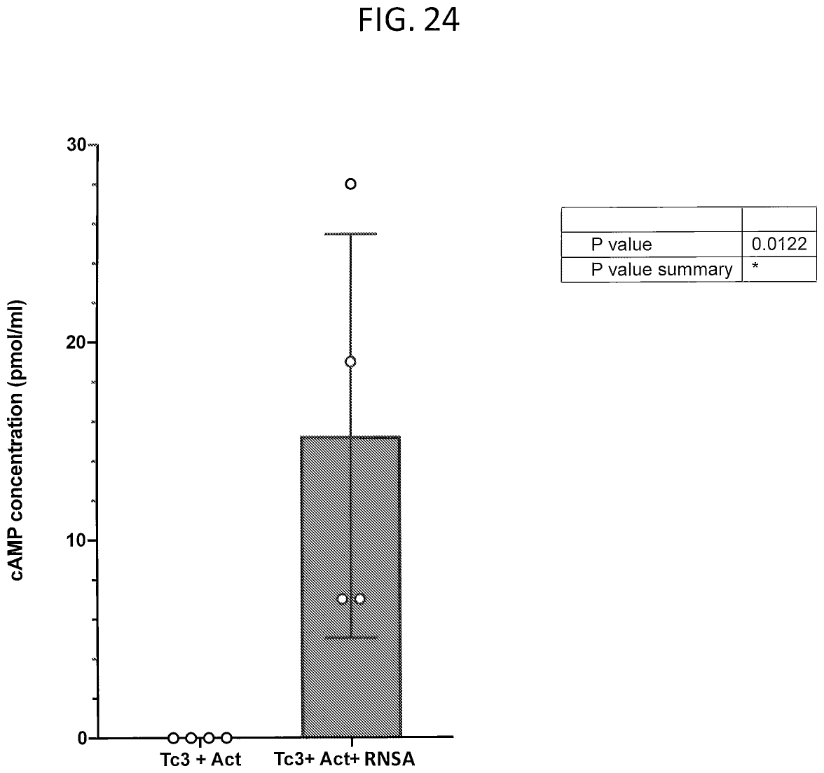

Next, the effect of the RNSA composition on the production of cAMP by activated T cells was determined. FIG. 24 shows that the RNSA composition promotes/induces cAMP production by activated T cells.

### Characterization of Eicosanoid Compounds in RNSA Compositions

Various samples were analyzed by mass spectrometry to determine the identity and concentrations of eicosanoid compounds in the RNSA composition. The analyzed samples included three samples extracted in αMEM media (αMEM-1, αMEM-2, and αMEM-3) which served as negative controls. Four RNSA composition samples produced using the cultured hMSCs described in Example 2 (BR-1, BR-2, BR-3, and BR-4) and three RNSA composition samples produced using the amniotic membrane extraction protocol described in Example 1 (CM-1, CM-2, and CM-3) were also analyzed.

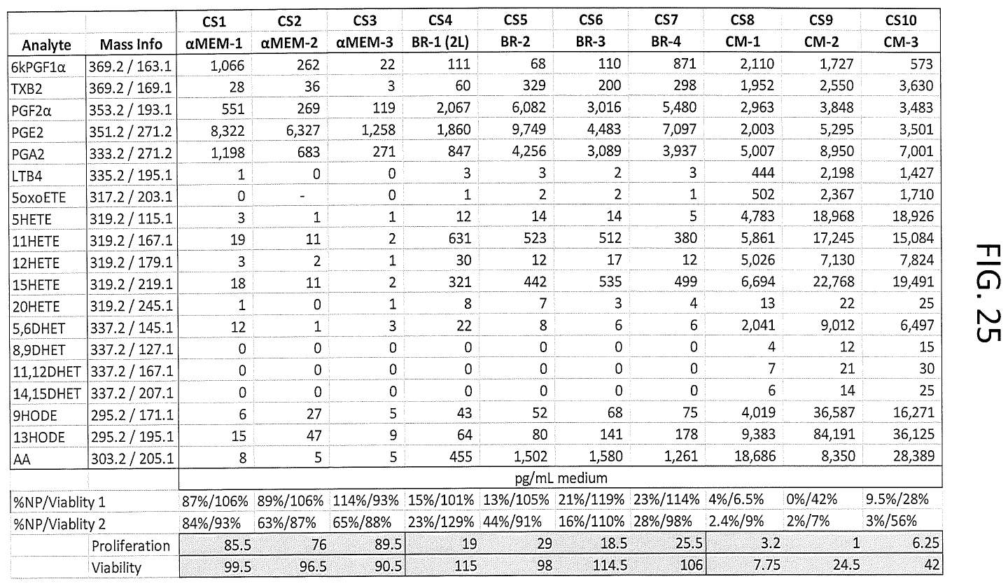

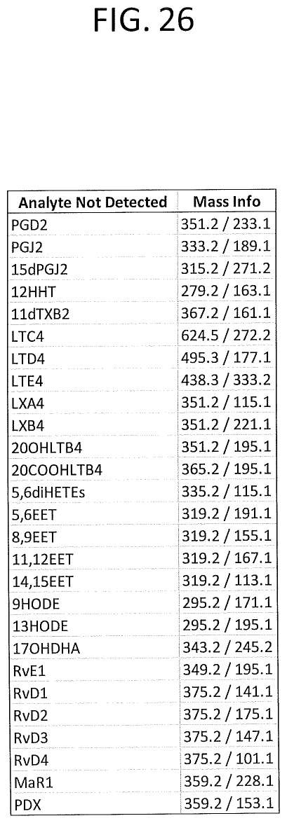

Results of the eicosanoid analysis are shown in FIG. 25. The concentration of each eicosanoid shown in the Analyte column was determined and is shown in pg/mL media. The analyzed compositions were also tested in the T cell proliferation and viability assays described above, the results of which are shown in the bottom two rows, respectively, of FIG. 25. It is clear from this analysis that the CM-1, CM-2, and CM-3 RNSA compositions are enriched for several eicosanoids compared to the αMEM and BR samples. Further, the CM samples inhibited T cell proliferation and reduced T cell viability, whereas the BR samples inhibited T cell proliferation to a lesser extent and did not reduce T cell viability. Several eicosanoid analytes were not detected in any of the analyzed samples, which compounds are shown in FIG. 26.

### Cell Proliferation as a Model for Regeneration

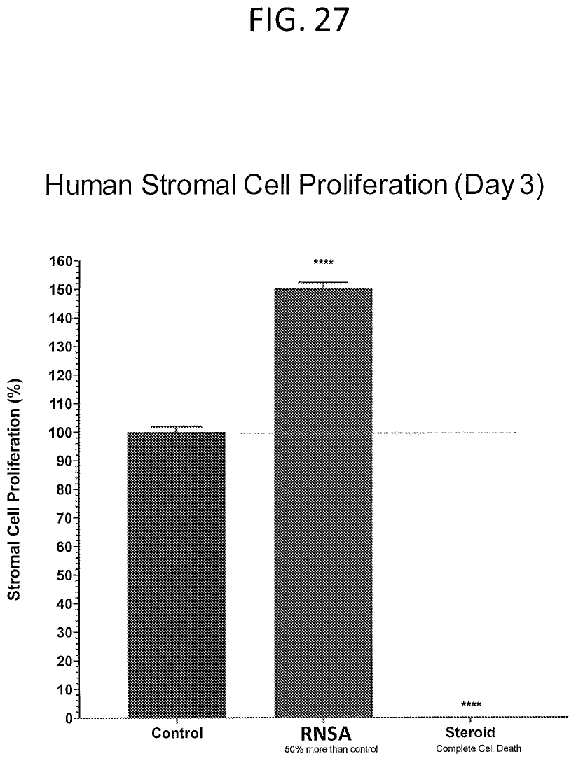

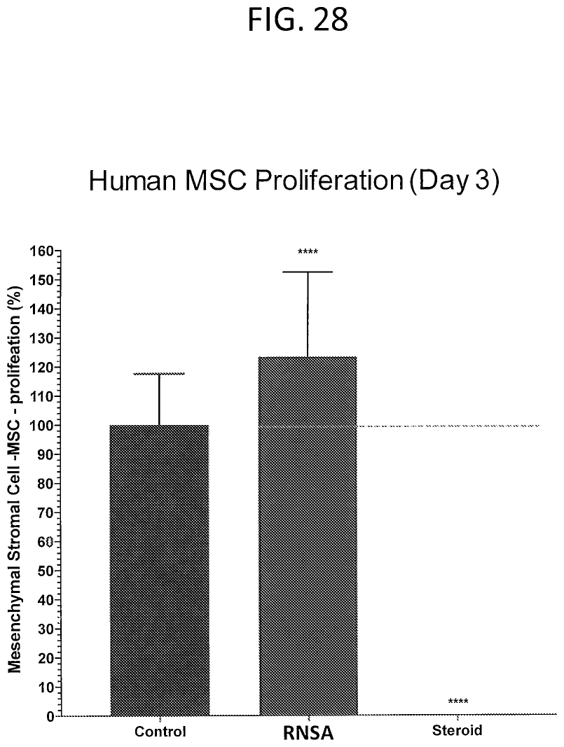

In order to test the regenerative properties of the RNSA composition, human stromal cells and human mesenchymal stromal cells (hMSC) were cultured in the absence or presence of the RNSA composition or in the absence or presence of a steroid. As shown in FIG. 27 and FIG. 28, the RNSA composition promotes proliferation of human stromal cells and hMSCs, respectively, whereas the steroid is completely cytotoxic.

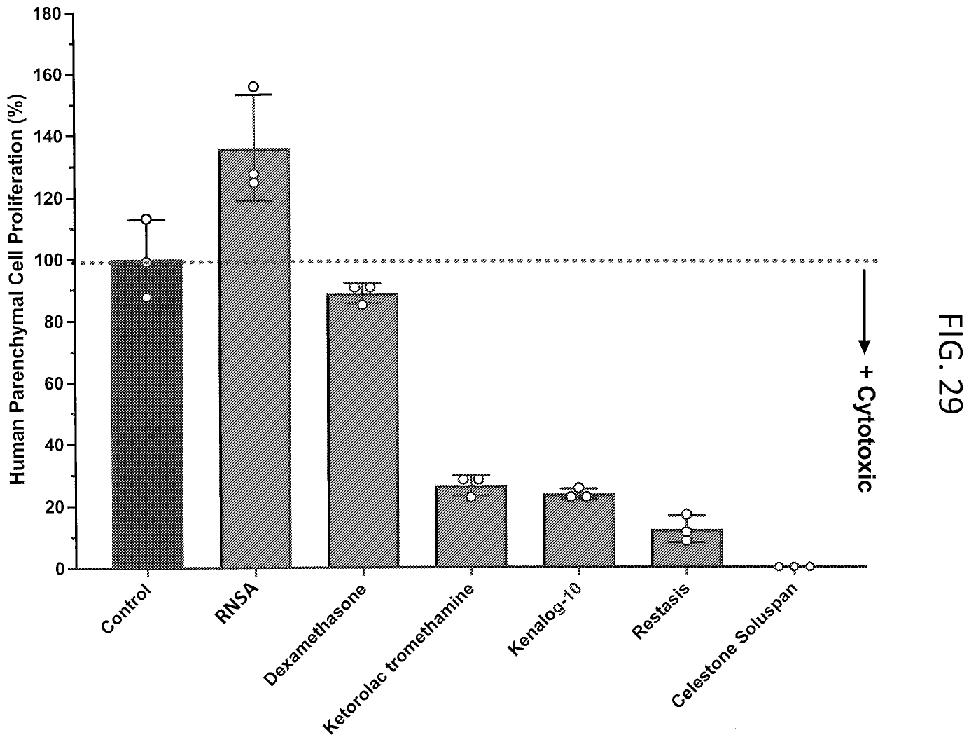

Similarly, the regenerative properties of the RNSA composition were tested using human parenchymal cells. As shown in FIG. 29, the RNSA composition promotes proliferation whereas the tested commercial compounds were each cytotoxic to varying degrees.

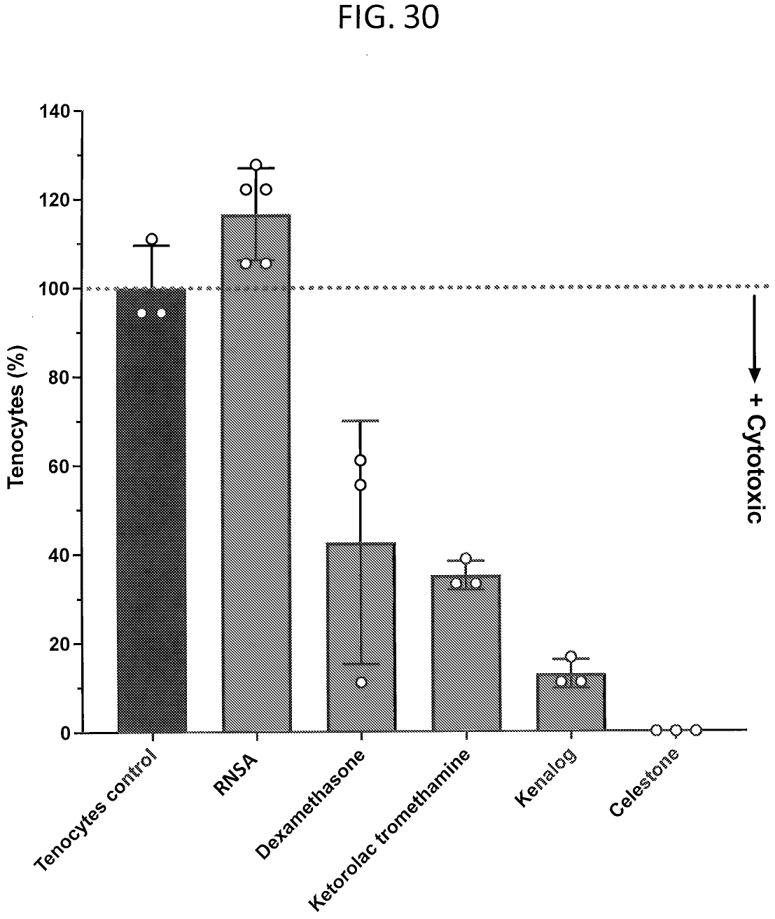

Similarly, the regenerative properties of the RNSA composition were tested using human tenocytes. As shown in FIG. 30, the RNSA composition promotes proliferation whereas the tested commercial compounds were each cytotoxic to varying degrees.

### Example 4: Evaluation of the Effect of Cell-Free Regenerative Nonsteroidal Anti-Inflammatory (RNSA) Compositions on a Model of Humanized Graft Versus Host Disease

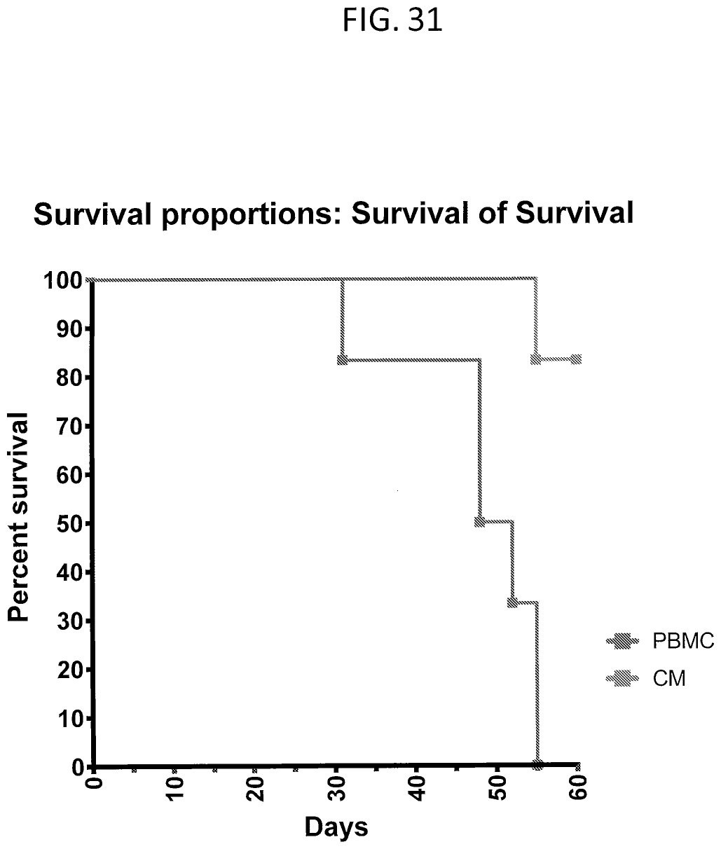

A humanized mouse model of Graft versus Host Disease (GvHD) was used to evaluate the effect of the RNSA composition. Briefly, the mice lacking an immune system are injected with human PBMCs, which begin attacking the murine tissues. As shown in the survival curve in FIG. 31, by day 50, half of the control mice injected only with media (PBMC) have died. In contrast, administration of the cell-free regenerative nonsteroidal anti-inflammatory composition (CM) rescues the GvHD mice such that by day 50, 100% of the treated mice are still alive.

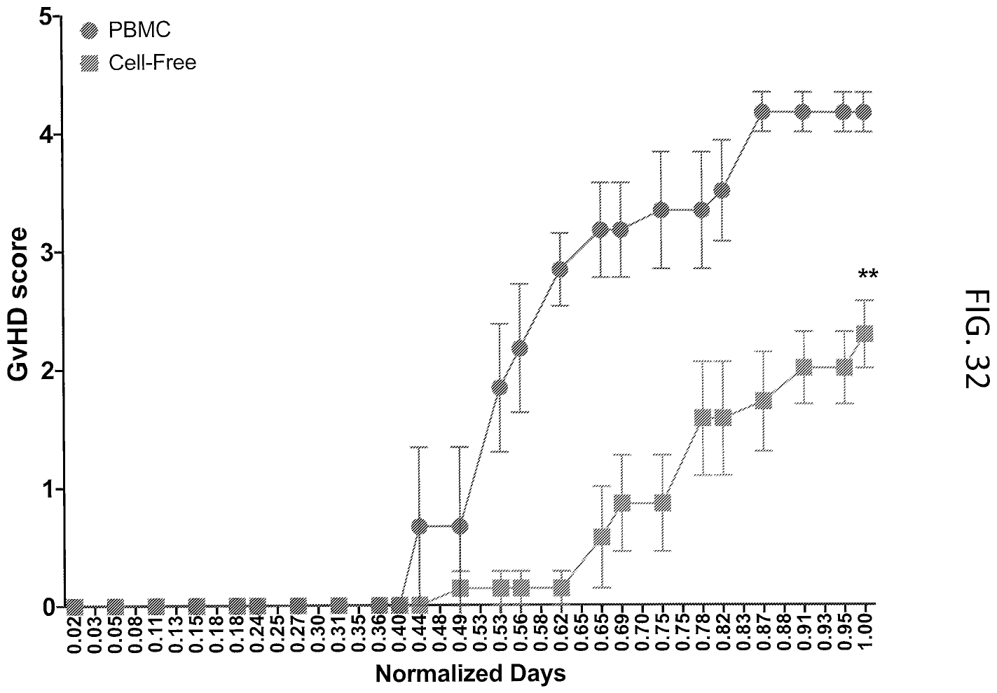

FIG. 32 is a plot of the GvHD score versus normalized days for the GvHD mice injected with media (PBMC) or with an exemplary cell-free regenerative nonsteroidal anti-inflammatory composition (Cell-Free) according to the invention. A higher GvHD score represents a poorer prognosis.

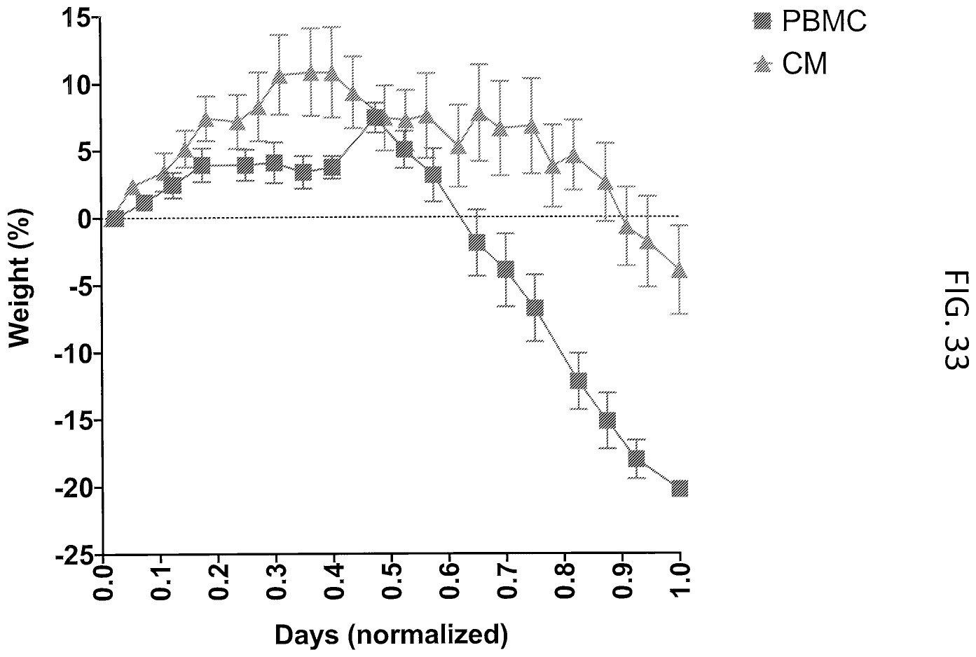

FIG. 33 is a plot of the body weight GvHD mice injected with media (PBMC) or with an exemplary cell-free regenerative nonsteroidal anti-inflammatory composition (CM) according to the invention. A drop in weight reflects the severity of the autoimmunity progression of the disease. The RNSA composition has a strong effect in ameliorating the severity of GvHD.

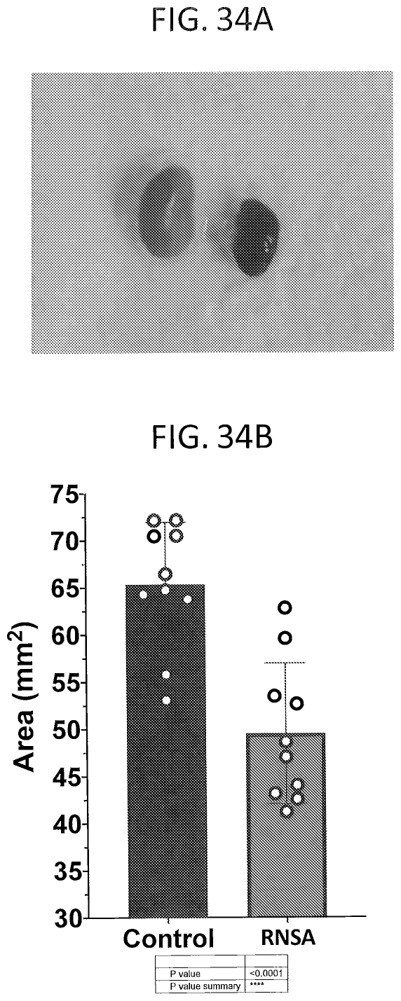

FIG. 34A is a photo of GvHD mouse model kidneys. The kidney on the left from the control mouse is enlarged indicating it is inflamed and likely fibrotic, whereas the kidney on the right is from the GvHD mouse treated with the RNSA composition. The kidney on the right looks normal. FIG. 34B is a plot of the GvHD mouse kidney areas in mm².

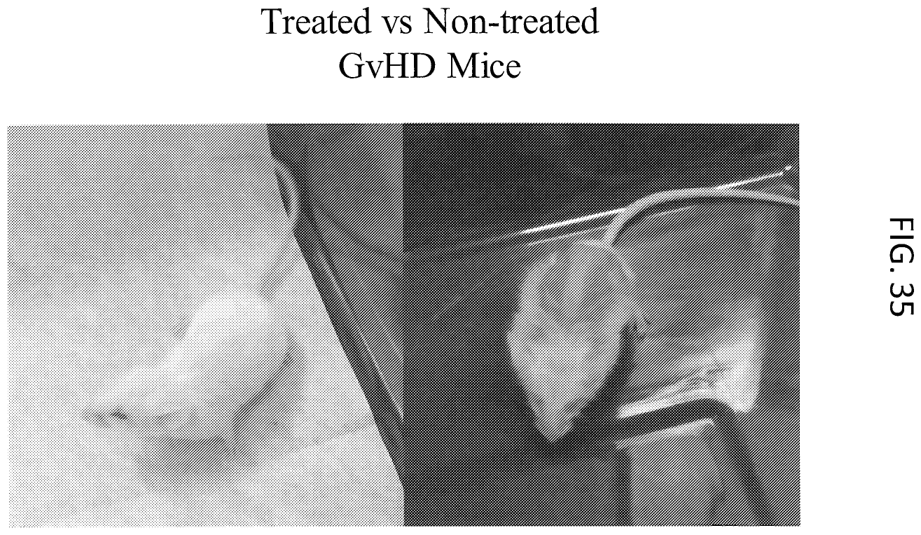

FIG. 35 is a photo of the GvHD mice. A GvHD mouse treated with an exemplary RNSA composition according to the invention is shown on the left whereas an untreated GvHD mouse is shown on the right.

### Example 5: Evaluation of the Effect of Cell-Free Regenerative Nonsteroidal Anti-Inflammatory (RNSA) Compositions on Cardiomyopathy

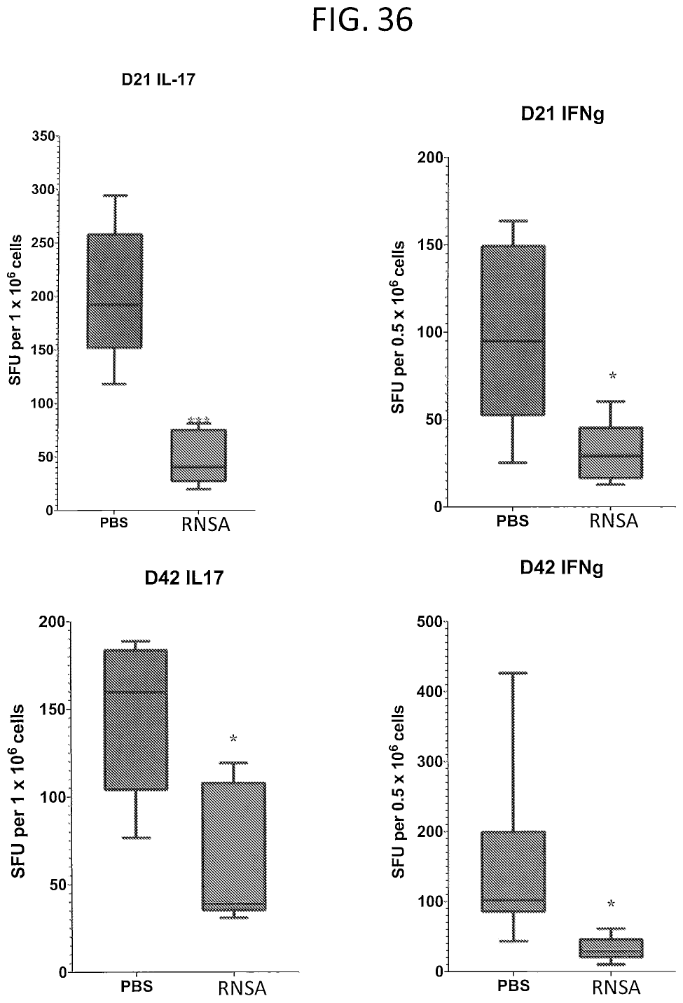

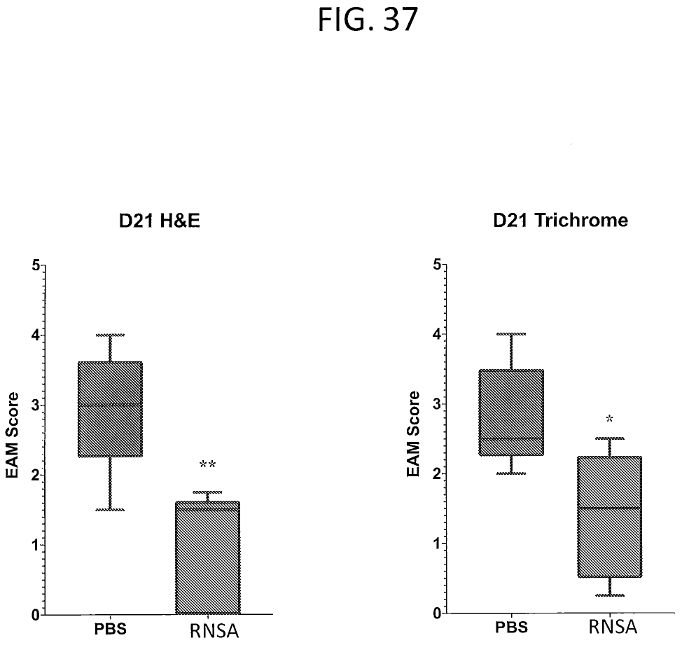

In a mouse model of cardiomyopathy (EAM), the RNSA composition reduced inflammatory markers IL-17 and IFNγ in vivo at Day 21 and Day 42 as determined using an ELISpot assay compared to the untreated control mouse (PBS) (FIG. 36). The RNSA composition also reduced inflammatory markers histopathology disease scores and Trichrome fibrosis scores at Day 21 compared to the untreated control mouse (FIG.

### Example 6: Evaluation of the Effect of Cell-Free Regenerative Nonsteroidal Anti-Inflammatory (RNSA) Compositions on Eczema

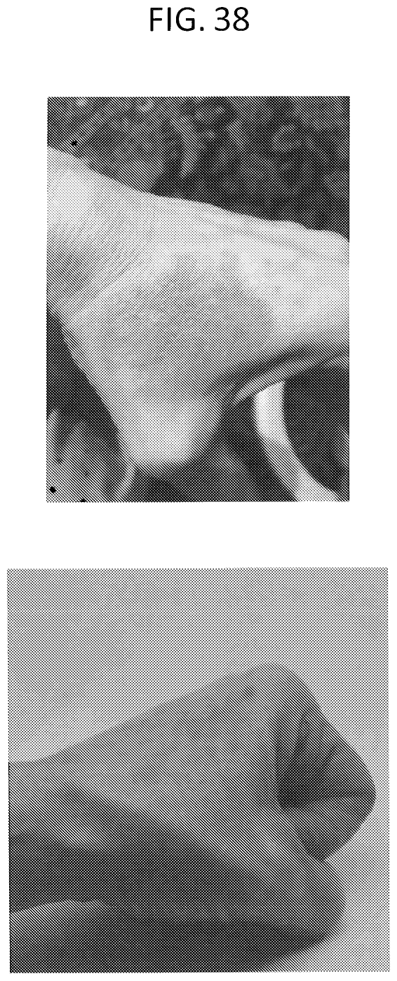

A human patient having eczema which was non-responsive to traditional treatment was treated with a first subcutaneous injection dose of an exemplary RNSA composition at the site of eczema (patient's left hand) on Day 1 and with a second subcutaneous injection dose on Day 30. FIG. 38 shows the patient's left hand on Day 1 and on Day 60. After 60 days, the patient's eczema was completely cleared.

### Example 7: Evaluation of the Effect of Cell-Free Regenerative Nonsteroidal Anti-Inflammatory (RNSA) Compositions on Tendinopathies

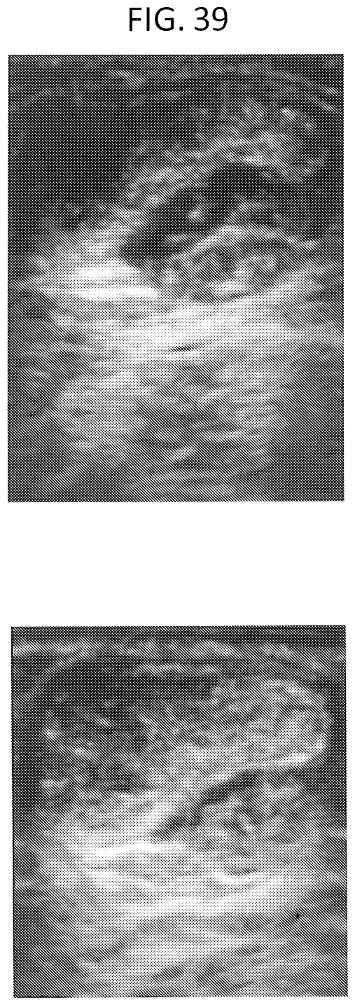

A human patient having tendinosis of the ankle and non-responsive to traditional treatments was treated with a single subcutaneous injection dose of an exemplary RNSA composition according to the invention at the site of tendinosis. FIG. 39 shows an ultrasound image of the tendinosis before treatment and an image of the healed tendon 30 days after treatment.

### Example 8: Evaluation of the Effect of Cell-Free Regenerative Nonsteroidal Anti-Inflammatory (RNSA) Compositions on COVID-19-Associated Pneumonia

Human patients having COVID-19-Associated Pneumonia are treated with the RNSA composition. Specifically, patients receive one dose of 1 mL of an exemplary RNSA composition according to the invention for each 10 kg of body weight into the blood stream via I.V. infusion. Patients are monitored by the attending physician(s) and hospital staff during the course of their hospital stay. Patients are evaluated by x-ray of the lungs at one month after the patient is discharged from the hospital. Patients receiving RNSA composition show a statistically significant improvement (i.e. decrease) of one or more symptoms of COVID-19-Associated pneumonia compared to patients receiving placebo.

### Example 9: Evaluation of the Effect of Cell-Free Regenerative Nonsteroidal Anti-Inflammatory (RNSA) Composition on Eyelid Sty

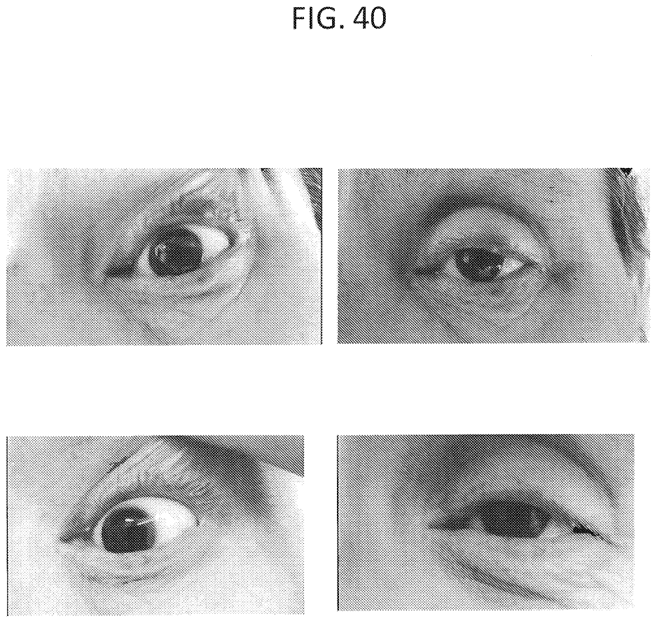

A human patient having an eyelid sty was treated with eye drops comprising an exemplary RNSA composition according to the invention. FIG. 40 shows two photos of the patient's eye having the eyelid sty prior to the treatment (top) and two phots of the same eye six weeks after treatment showing the eyelid had fully healed (bottom). The patient did not receive any other treatment or procedure for the eyelid sty.

We claim:

1. A cell-free or substantially cell-free regenerative nonsteroidal anti-inflammatory composition suitable for therapeutic or prophylactic use comprising a therapeutically or prophylactically effective amount of an isolated cell-free or substantially cell-free placenta-derived extract obtained from placental tissue from one or more mammalian donors wherein such tissue has naturally or been induced to undergo apoptosis or controlled cell death, wherein:
i. said extract comprises one or more eicosanoids optionally selected from 6kPGF1α, TXB2, PGF2α, PGE2, PGA2, LTB4, 5oxoETE, 5HETE, 11HETE, 12HETE, 15HETE, 20HETE, 5,6DHET, 8,9DHET, 11,12DHET, 14,15DHET, 9HODE, 13HODE, and AA;
ii. said composition is capable of inhibiting proliferation of activated T cells and is non-cytotoxic for one or more cells selected from stromal cells, mesenchymal stromal cells (MSCs), parenchymal cells, and tenocytes in a subject, in vivo, or in vitro.

2. The composition of claim 1, wherein:
(i) the placenta is selected from human, non-human primate, pig, sheep, horse, cow, dog, cat, rat, and mouse placenta, preferably human placenta;
(ii) the placental tissue is obtained from a single donor;
(iii) the placental tissue is obtained from more than one donor (pooled donor placental tissue sample);
(iv) the placenta comprises at least one placental tissue selected from amniotic membrane, chorion membrane, chorionic villus, umbilical cord, and Wharton's Jelly, preferably selected from at least one of amniotic membrane and/or chorion membrane'
(v) the at least one placental tissue comprises perinatal stromal cells (PSCs) and/or mesenchymal stromal cells (MSCs);
(vi) said composition is stable in solution at room temperature for at least eight weeks;
(vii) said composition is stable to lyophilization;
(viii) The composition of any one of the foregoing claims, wherein the T cells are CD4+, CD8⁺, CD4⁺/CD8⁺, CD11c⁺, CD11b⁺, and/or CD56+ T cells;
(ix) it is further capable of promoting proliferation of one or more cells selected from stromal cells, mesenchymal stromal cells (MSCs), parenchymal cells, and tenocytes in a subject, in vivo, or in vitro or
(x) any combination of (i) to (ix).

3. The composition of any one of the foregoing claims, which is:
(i) capable of reducing expression of one or more pro-inflammatory cytokines from activated peripheral blood mononucleated cells (PBMCs) and/or activated T cells in a subject, in vivo, or in vitro, optionally wherein the one or more pro-inflammatory cytokines is selected from TNFα, NFκB, IL-17A, II-6, and IFNγ;
(ii) capable of increasing cAMP production from activated T cells in a subject, in vivo, or in vitro;
(iii) modified by the addition of one or more other constituents, optionally non-actives or actives such as antibodies, cytokines, hormones, growth factors, drugs, antibiotics, analgesics, preservatives, pharmaceutically acceptable carriers and excipients, cells, e.g., autologous or allogeneic donor cells, e.g., immune cells, or any combination of the foregoing; or
(iv) any combination of the foregoing.

4. A method for producing a cell-free or substantially cell-free regenerative nonsteroidal anti-inflammatory composition, comprising:
i. obtaining at least one placental tissue from at least one mammal selected from human, non-human primate, pig, sheep, horse, cow, dog, cat, rat, and mouse, wherein the at least one placental tissue is selected from amniotic membrane, chorion membrane, chorionic villus, umbilical cord, and Wharton's Jelly, and wherein the at least one placental tissue comprises perinatal stromal cells (PSCs);
ii. optionally isolating the PSCs from said placental tissue and culturing the PSCs in at least one cell culture medium;
iii. permitting apoptosis of said placental tissue and PSCs comprised therein and/or permitting apoptosis of PSCs isolated therefrom to naturally occur and/or inducing apoptosis of said placental tissue and PSCs comprised therein and/or inducing apoptosis of PSCs isolated therefrom to produce an apoptotic extract; and
iv. separating the apoptotic extract or a portion thereof from the cells and tissue, for example, by decantation, centrifugation, and/or filtration;
thereby producing the cell-free or substantially cell-free regenerative nonsteroidal anti-inflammatory composition.

5. The method of claim 4, wherein:
(i) the PSCs comprise mesenchymal stromal cells (MSCs);
(ii) the mammal is a human or non-human primate;
(iii) the method further comprises conducting one or more screening assays to assess the effects of the isolated apoptotic extract or one or more portions thereof on the proliferation of activated T cells and/or the proliferation of one or more cells selected from stromal cells, mesenchymal stromal cells (MSCs), parenchymal cells, and tenocytes and/or on the expression of pro-inflammatory cytokines and/or the expression of anti-inflammatory cytokines in a mammalian subject, in vivo, or in vitro.
(iv) different portions of the isolated apoptotic extract are screened in order to assess potency;
(v) inducing apoptosis comprises serum deprivation, nutrient deprivation, and/or hypoxia; or
(vi) any combination of (i) to (v).

6. The method of claim 4 or 5, wherein apoptosis is induced by the following:
i. contacting the placental tissue with a non-cell culture medium in a ratio ranging from about 1 mL non-cell culture medium per 1 gram of placental tissue to about 100 mL non-cell culture medium per 1 gram of placental tissue, preferably in a ratio of about 10 mL non-cell culture medium per 1 g of placental tissue; and
ii. incubating the placental tissue in the non-cell culture medium in an air-tight environment at a temperature ranging from about 4° C. to about 42° C., preferably at about 37° C., for about 2 days to about 12 days, preferably for about 10 days, wherein the incubating optionally comprises agitation, for example, at about 90 rpm.

7. The method of any one of claims 4-6, which further comprises isolating the placental tissue PSCs and culturing the PSCs in at least one cell culture medium prior to inducing apoptosis.

8. The method of any one of claims 4-7, wherein inducing apoptosis comprises:
i. replacing the at least one cell culture medium with a non-cell culture medium; and
ii. incubating the cultured PSCs in the non-cell culture medium in an air-tight environment at a temperature ranging from about 4° C. to about 42° C., preferably at about 37° C., for about 3 days to about 5 days, preferably for about 4 days, wherein the incubating optionally comprises agitation.

9. The method of any one of claims 4-8, wherein:
(i) the cultured PSCs are cultured to at least 80% confluence;
(ii) the non-cell culture medium comprises saline solution;
(iii) the non-cell culture medium comprises saline solution that comprises 0.9% NaCl;
(iv) the non-cell culture medium comprises saline solution that comprises phosphate-buffered saline (PBS);
(v) the air-tight environment prevents gas exchange, thereby inducing a hypoxic environment;
(vi) the method further comprises washing the placental tissue with phosphate-buffered saline (PBS) prior to inducing apoptosis;
(vii) the method further comprises mincing the placental tissue prior to inducing apoptosis;
(viii) the method further comprises washing the cultured MSCs with phosphate-buffered saline (PBS) prior to inducing apoptosis;
(ix) the method further comprises contacting the placental tissue with one or more antimicrobial agents;
(x) the centrifugation comprises centrifugation at about 10,000×g for about 30 minutes;
(xi) the filtration comprises filtration through a 0.45 μm membrane;
(xii) the filtration comprises filtration through a 0.2 μm membrane, i.e. sterile filtration;
(xiii) the filtration comprises filtration through a 30 KDa MWCO membrane, a 10 KDa MWCO membrane, a 5 KDa MWCO membrane, a 3 KDa MWCO membrane, and/or a 2 KDa MWCO membrane; or
(xiv) any combination of (i) to (xiii).

10. A cell-free or substantially cell-free regenerative nonsteroidal anti-inflammatory composition produced by the method of any one of claims 4-9.

11. The composition of any one of the foregoing claims, which:
comprises one or more eicosanoids optionally selected from 6kPGF1α, TXB2, PGF2α, PGE2, PGA2, LTB4, 5oxoETE, 5HETE, 11HETE, 12HETE, 15HETE, 20HETE, 5,6DHET, 8,9DHET, 11,12DHET, 14,15DHET, 9HODE, 13HODE, and AA;
(ii) is capable of inhibiting proliferation of activated T cells in a subject, in vivo, or in vitro, wherein the T cells are CD4⁺, CD8⁺, CD4⁺/CD8⁺, CD11c⁺, CD11b⁺, and/or CD56⁺ T cells;
(iii) is non-cytotoxic for one or more cell types selected from stromal cells, mesenchymal stromal cells (MSCs), parenchymal cells, and tenocytes, in a subject, in vivo, or in vitro;
(iv) is capable of promoting proliferation of one or more cell types selected from stromal cells, mesenchymal stromal cells (MSCs), parenchymal cells, and tenocytes, in a subject, in vivo, or in vitro;
(v) is capable of reducing expression of one or more pro-inflammatory cytokines from activated peripheral blood mononucleated cells (PBMCs) and/or activated T cells in a subject, in vivo, or in vitro;
(vi) the one or more pro-inflammatory cytokines is selected from TNFα, NFκB, IL17A, IL-6, and IFNγ;
(vii) is capable of increasing cAMP production from activated T cells in a subject, in vivo, or in vitro;
(viii) is stable in solution at room temperature for at least eight weeks.
(ix) is stable to lyophilization;
(x) is modified by the addition of one or more other constituents, optionally non-actives or actives such as antibodies, cytokines, hormones, growth factors, drugs, antibiotics, analgesics, preservatives, pharmaceutically acceptable carriers and excipients, cells, e.g., autologous or allogeneic donor cells, e.g., immune cells, or any combination of the foregoing; or
(xi) any combination of (i) to (x).

12. A method of treatment or prevention of at least one inflammatory condition or disease or at least one symptom associated therewith, comprising administering a therapeutically or prophylactically effective amount of the cell-free or substantially cell-free regenerative nonsteroidal anti-inflammatory composition of any one of the foregoing claims to a subject in need thereof.

13. The method of treatment or prevention of any one of the foregoing claims, wherein the at least one inflammatory condition or disease is an acute or chronic condition associated with inflammation, e.g., an acute or chronic autoimmune disease associated with acute or chronic inflammation, optionally a viral or bacterial or fungal infection associated with acute or chronic inflammation, further optionally a hepatitis virus, ZIKA virus, herpes, papillomavirus, influenza virus, or coronavirus, further optionally COVID-19 or SARS.

14. The method of treatment or prevention of any one of the foregoing claims, wherein the at least one inflammatory condition or disease is an acute inflammatory condition or disease optionally a viral infection associated with acute inflammation, further optionally a coronavirus infection, e.g., COVID-19 or SARS.

15. The method of treatment or prevention of any one of the foregoing claims, wherein the at least one inflammatory condition or disease is selected from pneumonia, single or multiple organ failure or dysfunction, sepsis, cytokine storm, fever, neurological dysfunction or impairment, loss of taste or smell, cardiac dysfunction, pulmonary dysfunction, liver dysfunction, acute or chronic respiratory dysfunction, graft versus host disease (GVHD), cardiomyopathy, vasculitis, fibrosis, ophthalmic inflammation, dermatologic inflammation, gastrointestinal inflammation, tendinopathies, allergy, asthma, glomerulonephritis, pancreatitis, hepatitis, inflammatory arthritis, gout, multiple sclerosis, psoriasis, Acute Respiratory Distress Syndrome (ARDS), wound healing, diabetic ulcers, non-healing wounds, lupus, and other autoimmune diseases associated with acute or chronic inflammation.

16. The method of treatment or prevention of any one of the foregoing claims, wherein the symptoms associated with the inflammatory condition include one or more of pneumonia, cytokine storm, single or multiple organ failure, fibrosis, impaired respiratory function such as acute or chronic respiratory distress syndrome, fever, impaired cardiac function, impaired lung function, impaired liver function, impaired taste or smell, and impaired neurological function.

17. The method of treatment or prevention of any one of the foregoing claims, wherein the subject has pneumonia, optionally Covid-19-associated pneumonia and/or a pneumonia associated with another virus, e.g., influenza or another coronavirus, and/or a pneumonia associated with a fungus or bacterium.

18. The method of treatment or prevention of any one of the foregoing claims, wherein the ophthalmic inflammation comprises one or more of corneal regeneration, corneal wound healing, corneal melting, dry eye, ocular infection, eyelid sty, and autoimmune-associated peripheral ulcerative keratitis.

19. The method of treatment or prevention of any one of the foregoing claims, wherein the fibrosis comprises one or more of pulmonary fibrosis, cystic fibrosis, idiopathic pulmonary fibrosis, interstitial pulmonary fibrosis, radiation-induced lung injury, liver fibrosis, bridging fibrosis of the liver, cirrhosis, glial scar, arterial stiffness, arthrofibrosis, Crohn's disease, Dupuytren's contracture, keloid fibrosis, Mediastinal fibrosis, Myelofibrosis, Myocardial fibrosis, Peyronie's disease, Nephrogenic systemic fibrosis, Progressive massive fibrosis, pneumoconiosis, Retroperitoneal fibrosis, stromal fibrosis, Scleroderma, systemic sclerosis, chronic obstructive pulmonary disease (COPD), asthma, and adhesive capsulitis.

20. The method of treatment or prevention of any one of the foregoing claims, wherein the gastrointestinal inflammation comprises one or more of inflammatory bowel disease (IBD), Crohn's disease, ulcerative colitis, irritable bowel syndrome (IBS), and Celiac disease.

21. The method of treatment or prevention of any one of the foregoing claims, wherein the ophthalmic inflammation is associated with keratoconjunctivitis sicca.

22. The method of treatment or prevention of any one of the foregoing claims, wherein the dermatologic inflammation comprises eczema or psoriasis.

23. The method of treatment or prevention of any one of the foregoing claims, wherein the at least one autoimmune disease is selected from the group consisting of Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome, (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, ‘Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndrome type I, Polyglandular syndrome type II, Polyglandular syndrome type III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, and Vogt-Koyanagi-Harada Disease.

24. The method of treatment or prevention of any one of the foregoing claims, wherein:
(i) the effective amount comprises one or more doses of the composition, wherein each dose ranges from 0.1 mL/10 kg body weight to 10 mL/10 kg body weight, preferably 1 mL/10 kg body weight;
(ii) the composition is administered by one or more of injection, optionally intravenous (IV) or subcutaneous (SC) administration, nebulization, and eye drops;
(iii) the subject is selected from a human, non-human primate, pig, sheep, horse, cow, dog, cat, rat, and mouse, preferably human;
(iv) it further comprises the administration of at least one other active, e.g., an anti-inflammatory agent such as an anti-inflammatory antibody or anti-inflammatory fusion protein, an antiviral agent, an antibacterial agent, an antifungal agent, an analgesic, an anti-congestive agent, an anti-fever agent, or a combination of any of the foregoing;
(v) the subject has been diagnosed with or is suspected of having a coronavirus infection, optionally COVID-19;
(vi) the subject has been diagnosed with a coronavirus infection, optionally COVID-19, and is on a respirator, has Acute Respiratory Distress Syndrome (ARDS), and/or is experiencing respiratory difficulties;
(vii) the subject has been diagnosed with or suspected of having a coronavirus infection, optionally COVID-19, and optionally the subject comprises one or more risk factors that place the subject at higher risk for morbidity or a poor treatment outcome, e.g., age over 55 years, obesity, diabetes, cardiac problem or condition, respiratory condition, optionally asthma, COPD, cystic fibrosis, is a smoker, is a heavy drinker, has lupus, has elevated blood pressure, has cancer, receives chemotherapy, has (chronic) kidney disease and/or is on dialysis, or any combination of the foregoing; or
(viii) any combination of (i) to (vii).
